(19) United States
(12) Patent Application Publication
(10) Pub. No.: US 2009/0312676 A1
(43) Pub. Date: Dec. 17, 2009

(54) Metabolic Sink

(75) Inventors: Benny Rousso, Rishon-LeZion (IL); Shlomo Ben-Haim, London (GB); Yoram Izhaki, Moshav Kfar Hess (IL); Omer Einav, Emek Hefer (IL)

Correspondence Address: MARTIN D. MOYNIHAN d/b/a PRTSI, INC., P.O. BOX 16446, ARLINGTON, VA, 22215, US

(73) Assignee: Tylerton International Inc., Tortola (VG)

(21) Appl. No.: 12/223,398

(22) PCT Filed: Feb. 1, 2007
(86) PCT No.: PCT/IL2007/000136

§ 371 Date: Feb. 17, 2009

Related U.S. Application Data
(60) Provisional application No. 60/764,398, filed on Feb. 2, 2006.

(51) Int. Cl. A61F 7/12; A61F 7/00; A61H 1/00; A61N 1/36

(52) U.S. Cl. 601/15; 607/96; 607/112; 607/113; 607/105; 607/3

(57) ABSTRACT

A method for increasing metabolism of a subject's body in order to lose weight, comprising:
a) contacting a part of the body with a cooling element to remove heat from the body; and
b) repeating or continuing (a) so as to remove enough heat in total to lose at least 1 kilogram of body weight.

### RELATED APPLICATIONS

This application claims benefit under 119(e) of U.S. provisional patent application 60/764,398, filed Feb. 2, 2006, the disclosure of which is incorporated herein by reference.

### FIELD OF THE INVENTION

The present invention relates to increasing the metabolism rate by increasing heat loss from the body.

### BACKGROUND OF THE INVENTION

Obesity is becoming a widespread problem in developed countries. Some common solutions include exercising, dieting and stomach restriction.

Many persons, however, find it hard to stay with a regime of dieting or exercising.

Various drug treatments have been devised for treating obesity as well, for example, appetite suppressants, metabolic enhancements and drugs that prevent nutrient absorption. Such drugs have various side effects and none has shown significant success.

There are anecdotal reports that swimming in cold water increases heat loss and causes the body to increase metabolic output. Under conditions of cold, the body applies various physiological mechanisms to maintain body temperature. It is fairly well understood that during exposure to the cold, shivering can cause up to a 2.5 times increase in metabolic rate in an effort to maintain core temperature. This figure is mentioned in an article by Ellen Glickman-Weiss, “Fat Loss in the Cold?” on the website of VirtualMuscle.com, and is based on an article by I. Jacobs, L. Martineau, and A. L. Vallerand, “Thermoregulatory Thermogenesis of Humans During Cold Stress,” in Exercise and Sports Science Reviews, 1993.

While it has been suggested that a person sit lightly dressed in a cold room in order to lose weight, such schemes usually fail due to the pain caused by such cold, among other reasons.

Glacier Tek, Inc., of West Melbourne, Fla., sells “cool jackets” and “cool vests” in which a packet of material with a moderately cool melting point, about 15 degrees Celsius, is frozen in a refrigerator, and then held against the body in a jacket or vest to cool the body. The company suggests use of the product for people who work in hot environments, and to relieve symptoms of certain medical conditions.

Astronauts and divers wear suits with pumps which circulate water around the suit for heating and cooling, and use feedback to control the temperature inside the suit. Such cooling suits are described, for example, by P. Webb et al, “Heat regulation during exercise with controlled cooling,” Eur. J. Appl. Physiol. 62:193-197 (1991). The U.S. Army has also developed cooling suits for use by soldiers, as described in Release No. 01-40 from the Public Affairs Office of the U.S. Army Soldier & Biological Chemical Command, U.S. Army Soldier Systems Center, Natick, Mass., Jul. 9, 2001.

“Cold blankets” are used to reduce the core body temperature of patients before undergoing open heart surgery, as well as stroke patients in some circumstances.

The announcement by the National Institutes of Health Clinical Center of a clinical trial, “Body Heat Content and Dissipation in Obese and Normal Weight Adults,” ClinicalTrials.gov Identifier NCT00266500, Nov. 29, 2005, sponsored by the National Institute of Child Health and Human Development (NICHD), describes the use of a cooling device applied to the hand, thighs, or abdomen, held in place by a gentle vacuum, as well as other methods of cooling such as immersion in cool water, or a cool water spray. The purpose of the study is to provide preliminary evidence for future studies that will attempt to facilitate weight loss in obese subjects through guided applications of heat management.

W. J. O'Hara, C. Allen, and R. J. Shephard, “Treatment of obesity by exercise in the cold,” Can. Med. Assoc. J. 117:773-8, 786 (1977), and R. J. Shephard, Can. J. Sport Sci. 17:83-90 (1992), “Fat metabolism, exercise and the cold,” describe using a combination of exercise and exposure to the cold to treat obesity. R. J. Shephard, “Adaptation to exercise in the cold,” Sports Med. 2:59-71 (1985) finds that a combination of moderate exercise and facial cooling induces substantial fat loss over a one to two week period, and proposes possible mechanisms, including the possibility of increased resting metabolism. J. Arnold and D. Richard, “Exercise during intermittent cold exposure prevents acclimation to cold rats,” J. Physiol. 390:45-54 (1987), find that sedentary rats, but not exercising rats, develop an increase in base metabolism when exposed to cold.

A number of studies have been done to elucidate the thermoregulatory mechanisms in humans. A. V. Desruelle and V. Candas, “Thermoregulatory effects of three different types of head cooling in humans during a mild hyperthermia,” Eur. J. Appl. Physiol. 81:33-39 (2000), find that sweating rates are regulated by the core thermal inputs to the hypothalamic regulatory system, as indicated by tympanic temperature measurements. M. Cabanac and M. Caputa, “Open loop increase in trunk temperature produced by face cooling in working humans,” J. Physiol. 289:163-174 (1979) found evidence that brain temperature, rather than esophageal temperature, is precisely regulated during exercise, with the brain being cooled directly by cool blood returning from the face, when the face is cooled. In addition to the central hypothalamic thermoregulatory mechanism, there are also local thermoregulatory mechanisms in the skin, described for example in Mayo Clin. Proc. 78:603-612 (2003).

G. E. Alvarez, et al, “Relative roles of local and reflex components in cutaneous vasostriction during skin cooling in humans,” J. Appl. Physiol. 100: 2083-2088 (2006), published after the filing date of U.S. provisional patent application 60/764,398, examines the effects of local and whole body cooling on vasostriction. Alvarez et al used metal-Peltier cooler-heater probes, as well as water-perfused cooling suits.

Vasodilation can be artificially produced by a variety of means, including vacuum (D. Grahn et al, J. Appl. Physiol. 85:1643-1648 (1998)), electricity (M. Tartas et al, J. Appl. Physiol. 99: 1538-1544 (2005)), topical ointments containing methyl nicotinate (C. M. Kesick et al, Technical Report T-01/8, April 2001, U.S. Army Research Institute of Environmental Medicine, Natick, Mass.), or nitroglycerin (S. B. Robinson et al, Technical Report, Accession Number ADA387041, January 2001, U.S. Army Research Institute of Environmental Medicine, Natick, Mass.), and acoustic energy (WO 2004/071570 to Horzewski et al). Tartas et al found that electrically induced vasodilation is greater if electric current is applied intermittently, with an interval of 10 minutes or more between applications of current, and that electrically induced vasodilation is inhibited by aspirin, suggesting that prostaglandins play a role in the vasodilation. They also found that local effects are different at the anode and the cathode, for direct current. Ootsuka and Blessing, in a paper in the Journal of Physiology appearing online at http://jp.physoc.org/cgi/content/abstract/jphysiol.2003.048041v1, downloaded on Jan. 4, 2007, found that activation of 5-Hydroxytryptamine 1A receptors, by intravenous injection of various chemical agonists, can inhibit cold-induced cutaneous vasoconstriction in rabbits, leading to greater loss of heat during exposure to cold.

The disclosures of the documents cited above are incorporated herein by reference.

### SUMMARY OF THE INVENTION

An aspect of some embodiments of the invention relates to increasing a body metabolism of a subject by increasing a loss of heat from the body. In some embodiments of the invention, the metabolic increase is utilized to effect a weight loss, for example of at least half a kilogram, or at least one kilogram, or at least 2 kilograms, or at least 5 kilograms. In an exemplary embodiment of the invention, heat loss is increased by causing vasodilatation, optionally on an exposed body part. In an exemplary embodiment of the invention, heat loss is increased by contacting parts of the body with a cooling element, adapted to remove heat from the body. Optionally, the rate at which the cooling element removes heat from the body is actively controlled while the cooling element remains in contact with the body, and not simply by removing the cooling element from the body or reducing the contact area. Optionally, heat is removed in a manner which avoids or reduces pain or discomfort to the subject. Optionally, heat is removed in a manner which prevents and/or overcomes vasoconstriction. Optionally the heat is removed without lowering the core body temperature significantly, for example without lowering it by more than 1 degree Celsius, or by more than 0.5 degrees, or by more than 0.2 degrees.

In an exemplary embodiment of the invention, the cooling element is cooled continuously, for example by fluid flowing through it, while it is removing heat from the body. Additionally or alternatively, the cooling element is cooled before being used, for example by refrigerating it, and uses its heat capacity to remove heat from the body.

In an exemplary embodiment of the invention, heat loss is sustained over considerable periods of time, desirably without tissue damage. Optionally, heat loss is sustained over a long enough time, at a high enough rate, and/or at a high enough duty cycle, so that the base metabolism of the subject increases, and remains elevated even when the subject is not being cooled. Evidence for such an effect is described in the papers by Shephard (1985) and by Arnold and Richard (1987), cited above in the Background section.

In an exemplary embodiment of the invention, heat loss is local, for example, through one or both hands, legs or an abdomen, and is accomplished, for example, using a device located under clothing or serving as a separate article of clothing or jewelry. Optionally, the device is comfortable and attractive enough to wear during waking hours, and/or in public, and/or the device substantially does not interfere with normal activities. Additionally or alternatively, the device is worn while sleeping, and substantially does not interfere with sleep. Optionally, the device is part of or mounted on a piece of furniture, such as a chair or a bed, and is used with the subject sitting or lying in it, or on it. Optionally, the device is part of a piece of exercise equipment, for example weights, or the seat of an exercise bicycle, and removes heat from the subject while the subject is exercising.

In an exemplary embodiment of the invention, cooling is applied to the body in a manner which fools the physiological vasoconstriction/vasodilatation mechanism and optionally prevents normal heat-retaining mechanisms from operating to their full capacity. Optionally, a relatively small amount of heat is applied to a part of the body, such as the head or face, which has a sensitive systemic effect on the body's thermoregulatory mechanism, allowing a greater amount of heat to be removed from other parts of the body, for example the trunk or limbs.

In an exemplary embodiment of the invention, heat-loss is under computer control, for example controlling periods of lower and higher temperature, applying above normal temperatures at times, and/or controlling a degree of temperature difference. One or more sensors may be provided as well, for example, shiver sensors, temperature sensors and/or blood flow sensors. Measurements from such sensors may be used to close a feedback loop. For example, negative feedback is used to maintain shivering at an amplitude that provides a desired tradeoff between an increase in metabolism and not causing too much discomfort. Additionally or alternatively, negative feedback is used to prevent or reduce vasoconstriction.

In an exemplary embodiment of the invention, a cooling element is applied and maintained at a temperature and/or has a contact configuration such that a person in contact with the element does not feel pain associated with cold, but rather, at most, a feeling of coolness.

In an exemplary embodiment of the invention, vasodilation is induced using chemical means, for example vasodilating bio-active materials. Optionally the vasodilation is local, produced for example by applying the materials topically.

In an exemplary embodiment of the invention, vasodilation is provided, optionally locally, by mechanical means, for example, periodic massage or vibration, sound waves, or partial vacuum, or by electric stimulation.

An aspect of some embodiments of the invention relates to a heat-sink device adapted for contact or close proximity with the human body and which feels cool, rather than cold to the touch. By feeling cool is meant that only a sensation of low temperature is felt, without an associated pain caused by the low temperature.

In an exemplary embodiment of the invention, the cool sensation is maintained by keeping the device at a temperature above the firing temperature of cold-pain sensing fibers.

In an exemplary embodiment of the invention, the cool sensation is maintained by temperature changes and/or invoking adaptation mechanisms of the body, which confuse the body cold-sensing mechanism.

In an exemplary embodiment of the invention, an anesthetic cream is used to deaden the feeling of cold.

In an exemplary embodiment of the invention, cold-pain is avoided by suitable placement of contact points between the device and the skin.

In an exemplary embodiment of the invention, the cooling element or heat-sink device is used internally, for example in the mouth, the nose, the ears, the rectum, or the vagina, or a cooling fluid or other material is introduced into the body through the esophagus, or through the rectum, or intravenously. Optionally, the cooling element or material has a purpose other than cooling, for example irrigation of the colon. Optionally, blood is cooled by applying the cooling element to blood vessels near the surface of the body, or by removing blood, cooling it, and introducing it back into the bloodstream.

There is thus provided, in accordance with an exemplary embodiment of the invention, a method for increasing metabolism of a subject's body in order to lose weight, comprising:
a) contacting a part of the body with a cooling element to remove heat from the body; and
b) repeating or continuing (a) so as to remove enough heat in total to lose at least 1 kilogram of body weight.

Optionally, the cooling element is worn by the subject.

Optionally, the cooling element is integrated with clothing.

Optionally, the cooling element is comprised in furniture used by the subject.

Optionally, the furniture conforms to the body of the subject.

Optionally, the cooling element is comprised in jewelry worn by the subject.

Optionally, the cooling element is comprised in exercise equipment, used by the subject for exercising while the cooling element removes heat from the subject's body.

In an embodiment of the invention, removing heat from the body is at a great enough rate, over a long enough period at a great enough duty cycle, so that the subject's base metabolism increases by at least 10% for at least one day after the heat is removed.

Optionally, contacting a part of the body comprises contacting an internal part of the body.

Optionally, the method also includes performing an action on the body other than cooling.

In an embodiment of the invention, the cooling element comprises fluid introduced into the subject's colon, and the action is colonic irrigation.

Optionally, contacting comprises exerting pressure on the body by the cooling element.

Alternatively or additionally, contacting comprising drawing the body and the cooling element together by vacuum.

Alternatively or additionally, contacting comprises contacting through a thermally conducting liquid.

Optionally, the cooling element removes at least 10 kilocalories of heat within a period shorter than one hour.

Optionally, the cooling element removes at least 100 kilocalories of heat within a period shorter than one hour.

Optionally, the cooling system does not lower the core body temperature by more than 1 degree Celsius when the heat is removed from the body.

Optionally, the method also includes actively controlling the rate of heat removal by the cooling element while it is in contact with the body, using a control algorithm.

In an embodiment of the invention, the method also includes contacting a second part of the body with a second element, and actively controlling the second element to remove heat from the body or to add heat to the body, using the control algorithm, wherein the control algorithm treats the cooling element and the second element differently.

Optionally, the heating or cooling the second part of the body has a systemic effect on the body's thermoregulatory mechanism.

Optionally, the second part of the body is at least part of the head.

Optionally, the method also includes choosing a tolerable level of discomfort, wherein actively controlling the rate of heat removal by the cooling element comprises actively controlling the rate of heat removal to avoid exceeding the tolerable level of discomfort.

Optionally, choosing the tolerable level of discomfort is done at least within limits by the subject, in real time during the removal of heat from the body.

Optionally, actively controlling the rate of heat removal comprises controlling the rate of heat removal to avoid damage to body tissue.

In an embodiment of the invention, the method also includes reducing vasoconstriction during the removal of the heat from the body.

Optionally, reducing vasoconstriction comprises causing vasodilation.

Additionally or alternatively, reducing vasoconstriction comprises indirectly stimulating a nerve.

Optionally, reducing vasoconstriction comprises applying a chemical to the skin.

Optionally, the chemical is an alpha blocker or a calcium channel blocker or both.

Optionally, reducing vasoconstriction comprises applying mechanical stimulation.

Optionally, the mechanical stimulation comprises a partial vacuum.

Additionally or alternatively, the mechanical stimulation comprises acoustic energy.

Optionally, reducing vasoconstriction comprises applying electrical stimulation.

Optionally, reducing vasoconstriction comprises heating the skin.

Optionally, heating the skin is alternated with removing the heat, in such a way that there is a net removal of heat from the body.

There is further provided, in accordance with an exemplary embodiment of the invention, a method for increasing metabolism of a body in order to lose weight, comprising:
a) causing vasodilation of peripheral blood vessels locally in a part of the body, thereby causing heat to be lost from the body, beyond the heat that would have been lost from the body in the same thermal environment in the absence of the vasodilation; and
b) repeating or continuing (a) so as to remove enough heat in total to lose at least 1 kilogram of body weight.

Optionally, the vasodilation causes at least 10 kilocalories of heat to be lost from the body within a period shorter than one hour.

Optionally, the vasodilation causes at least 100 kilocalories of heat to be lost from the body within a period shorter than one hour.

There is further provided, in accordance with an exemplary embodiment of the invention, a method for increasing metabolism of a subject's body in order for the subject to lose weight, the method comprising:
a) heating a part of the body, causing a thermoregulatory mechanism of the body to increase heat loss from another part of the body;
b) measuring at least an indicator of the increased heat loss; and
c) repeatedly or continuously adjusting the rate of heating in response to a change in the measured indicator, in a direction opposite to the change in indicated heat loss; wherein the increase in heat loss, averaged over a period of time, is greater than the rate of heating.

Optionally, heating part of the body causes the thermoregulatory mechanism to increase heat loss by increasing one or both of vasodilation and sweating.

Optionally, heating a part of the body comprises heating at least part of the head.

There is further provided, in accordance with an exemplary embodiment of the invention, a cooling system for increasing metabolism of a body in order to lose weight without exceeding a chosen tolerable level of discomfort, the system comprising:
a) a cooling patch adapted to directly or indirectly contact the skin on a part of the body, which cooling patch removes heat from the body by cooling the skin; and
b) a controller adapted to actively control the rate at which the cooling patch removes heat from the body while it is in contact with a given area of the skin; wherein the controller is adapted to control the cooling patch to remove the heat from the body using a feedback loop that regulates the rate of heat loss, or the level of discomfort, or both.

Optionally, the cooling patch is adapted to be worn by the subject.

Optionally, the cooling patch is integrated with clothing.

Alternatively or additionally, the cooling patch is comprised in furniture.

Optionally, the furniture is adapted to conform to the body of the subject.

Alternatively or additionally, the cooling patch is comprised in jewelry.

Alternatively or additionally, the cooling patch is comprised in exercise equipment.

Optionally, the feedback loop regulates the rate of heat loss to be at least 10 kilocalories in less than one hour.

Optionally, the feedback loop regulates the rate of heat loss to be at least 100 kilocalories in less than one hour.

Optionally, the controller is adapted to control the cooling patch to remove the heat without lowering the core temperature of the body by more than 1 degree Celsius.

Optionally, the cooling patch is comprised in a glove, adapted to contact the skin of the hand.

In an embodiment of the invention, the cooling system also comprises:
a) a cooling fluid; and
b) a pump which circulates the cooling fluid through the cooling patch, thereby removing the heat from the body.

Optionally, the cooling system also includes a detachable reservoir of a cooling material, adapted for cooling in advance, through which the cooling fluid also circulates.

Optionally, the cooling system also includes a refrigeration unit which cools the cooling fluid when the cooling fluid passes through it, and the pump also circulates the cooling fluid through the refrigeration unit, thereby transferring the heat removed from the body to the refrigeration unit.

In an embodiment of the invention, the cooling patch comprises at least one Peltier unit.

Optionally, the cooling system also includes moving elements adapted to cause vasodilation of the skin cooled by the cooling patch, by vibrating or massaging.

Optionally, the cooling system also includes electrodes and a source of electric current adapted to cause vasodilation of the skin cooled by the cooling patch, by electric stimulation.

In an embodiment of the invention, the cooling system also includes a heating element adapted to cause vasodilation of the skin cooled by the cooling patch, by heating the skin alternately with the cooling of the skin.

Optionally, the heating element comprises a source of heated fluid, and a pump which pumps the fluid through the cooling patch.

Optionally, the heating element comprises a Peltier unit.

In an embodiment of the invention, the controller is adapted to alternately control the cooling patch to cool the skin for a cooling interval, and control the heating element to heat the skin for a heating interval.

Optionally, the cooling interval and heating interval are fixed.

Optionally, the cooling system also includes at least one sensor, and one or both of the cooling and heating interval depend on data from the at least one sensor.

Optionally, the controller controls the cooling patch to cool the skin at a rate that varies in time.

Optionally, the cooling system also includes at least one sensor, and the rate is a function of time that depends on data from the at least one sensor.

Optionally, the at least one sensor senses a temperature of the skin.

Optionally, the at least one sensor senses a temperature of the cooling patch.

Optionally, the at least one sensor senses blood flow.

Optionally, the cooling system also includes a cooling fluid and a pump which circulates the cooling fluid through the cooling patch, thereby removing the heat from the body, and the at least one sensor senses a flow rate of the cooling fluid.

### BRIEF DESCRIPTION OF THE DRAWINGS

Exemplary embodiments of the invention are described in the following sections with reference to the drawings. The drawings are not necessarily to scale and the same reference numbers are generally used for the same or related features that are shown on different drawings.

FIG. 1 is a schematic view of body tissue being cooled by a cooling system in accordance with an exemplary embodiment of the invention;

FIGS. 2A-2E are schematic views of exemplary placement areas for a cooling patch used in a cooling system such as that shown in FIG. 1, or exemplary placement areas for a different type of cooling system, in accordance with an exemplary embodiment of the invention;

FIG. 2F is a schematic view of a colonic irrigation system which also acts as a cooling system, according to an exemplary embodiment of the invention;

FIGS. 3A, 3B and 3C are schematic views showing the circulation of fluid through cooling patches in accordance with three exemplary embodiments of the invention;

FIG. 4A is a schematic perspective view of a cooling system using Peltier elements, in accordance with an exemplary embodiment of the invention;

FIG. 4B is a schematic perspective view of a cooling module using a Peltier element, in accordance with an exemplary embodiment of the invention;

FIG. 4C is a schematic perspective view of a cooling surface, comprising an array of the cooling modules shown in FIG. 4B, together with fans for removing heat, in accordance with an exemplary embodiment of the invention;

FIG. 4D is a schematic perspective view of the array shown in FIG. 4C, incorporated into the seat of a chair, in accordance with an exemplary embodiment of the invention;

FIG. 5 is a flowchart of a feedback cooling method, in accordance with an exemplary embodiment of the invention;

FIG. 6 is a schematic view of a cooling device in the form of a glove, using vibrating or moving elements to cause vasodilation, according to an exemplary embodiment of the invention;

FIG. 7 is a schematic view of a cooling device in the form of a glove, using electrical stimulation to cause vasodilation, according to an exemplary embodiment of the invention;

FIG. 8 is a schematic view of a cooling device in the form of a glove, using a topical cream to cause vasodilation and/or to anesthetize the skin, according to an exemplary embodiment of the invention;

FIG. 9 is a schematic cross-sectional view of a subject's skin in contact with a cooling device, the inner surface of which exudes a chemical, in accordance with an exemplary embodiment of the invention;

FIG. 10 is a schematic cutaway view of a cooling device in the form of a glove, with four layers, in accordance with an exemplary embodiment of the invention;

FIG. 11 is a more detailed schematic cross-sectional view of part of the cooling device shown in FIG. 10;

FIGS. 12A, 12B, and 12C are graphs schematically showing skin temperature, blood flow, and heat loss rate as functions of time in a pulsed cooling system, according to an exemplary embodiment of the invention; and

FIG. 13 is a schematic view of a cooling system which alternately cools and heats the skin, and uses feedback to control cold and hot temperature and flow rate, according to an exemplary embodiment of the invention.

FIG. 14 shows a flowchart for a method of cooling and heating the body, according to an exemplary embodiment of the invention.

### DETAILED DESCRIPTION OF EXEMPLARY EMBODIMENTS

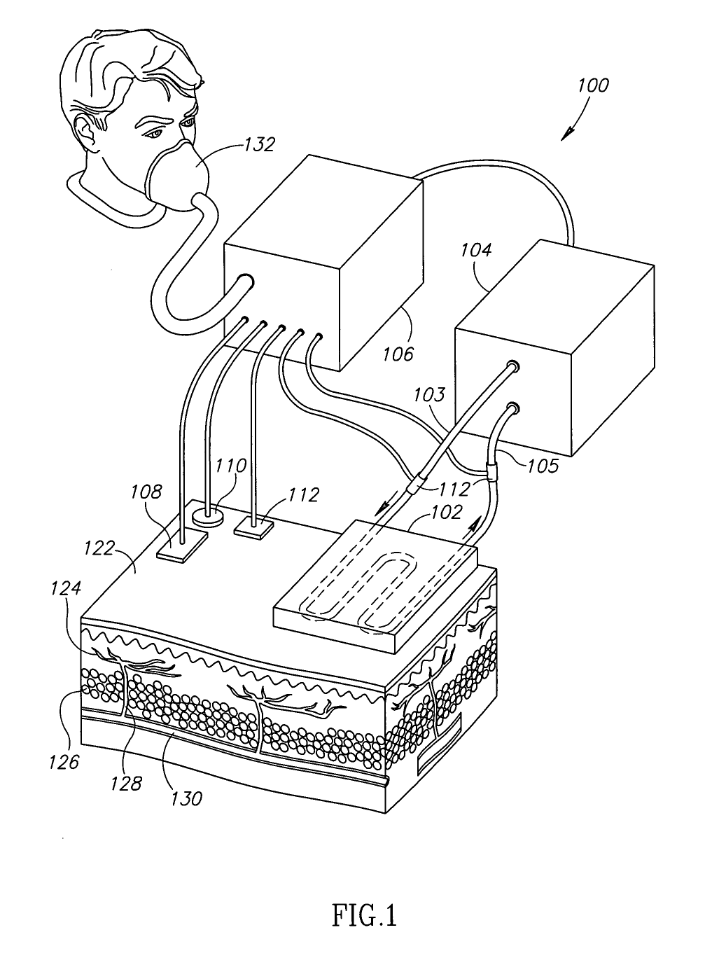

FIG. 1 schematically shows a cooling system 100, used to increase metabolism, and induce weight loss, by drawing heat out of the body, in accordance with an exemplary embodiment of the invention. A cooling patch 102, optionally has one or more tubes filled with water or another cooling fluid, for example another liquid or a gas, which circulates through cooling patch 102. The fluid is optionally cooled in a refrigerating unit 104, circulated by way of inlet tube 103 through cooling patch 102 where it is heated by the body, and returns by way of outlet tube 105 to refrigerating unit 104, where it is cooled down again. The fluid need not be cooled below the ambient temperature of the air, since heat is generally transferred from the skin to cooling patch 102 more efficiently than heat is lost from the skin to the air. Refrigerating unit 104 optionally cools the fluid using a conventional mechanical refrigeration cycle, expanding a gas (such as Freon or an environmentally safer substitute) adiabatically, then compressing it isothermally. Alternatively, refrigeration unit 104 uses evaporative cooling, or any other cooling method known in the art, including, for example, a solid state cooling method such as the Peltier method. Optionally, an existing household refrigerator or air conditioning unit is adapted to serve as refrigeration unit 104.

In some embodiments of the invention, the cooling fluid does not circulate back to refrigeration unit 104 from cooling patch 102, but only goes one way from refrigeration unit 104 to cooling patch 102. Pumping cooling fluid in only one direction may be particularly advantageous if air is used as the cooling fluid. Optionally, after passing through cooling patch 102, the air or other cooling fluid is released to the environment far enough away from cooling patch 102 so that little or none of the heat goes back into the body. In some embodiments of the invention, instead of the cooling fluid flowing through a tube in cooling patch 102, cooled air or another cooling fluid flows directly against the skin.

Whatever method is used for cooling, system 100, including refrigerating unit 104, is optionally portable, for example weighing less than 10 kg, or less than 5 kg, or less than 2 kg, and can be used by the subject at home, or at work, or during any normal activities. Optionally, refrigerating unit 104 is smaller than 50 cm across in its longest dimension, or smaller than 30 cm across, or smaller than 20 cm across. Optionally, cooling system 100 is powered by a battery or another energy storage device, for example a standard automotive battery, or a smaller, optionally more advanced battery, which is also portable, and which optionally can supply as much as 100 watts of electrical power for as much as one hour. Optionally, cooling system 100 is portable, but is plugged into a non-portable power source, for example a wall socket, when it is moved to a different location. Optionally, the cooling system can continue to operate, without electric power, for a period of time, by using a material which has been previously cooled, for example by detaching a reservoir of the material and storing it in a refrigerator or freezer when it is not being used, in order to cool the cooling fluid. Optionally the material has been previously frozen, and optionally it has a relatively high specific heat of fusion, and/or a freezing point somewhat below an operating temperature used for the cooling fluid.

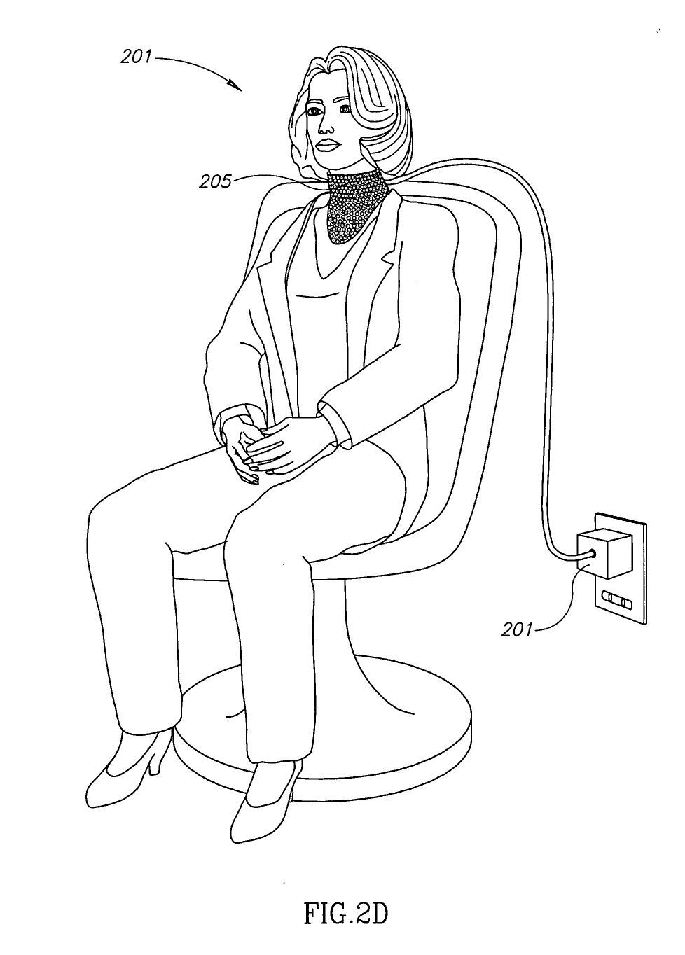

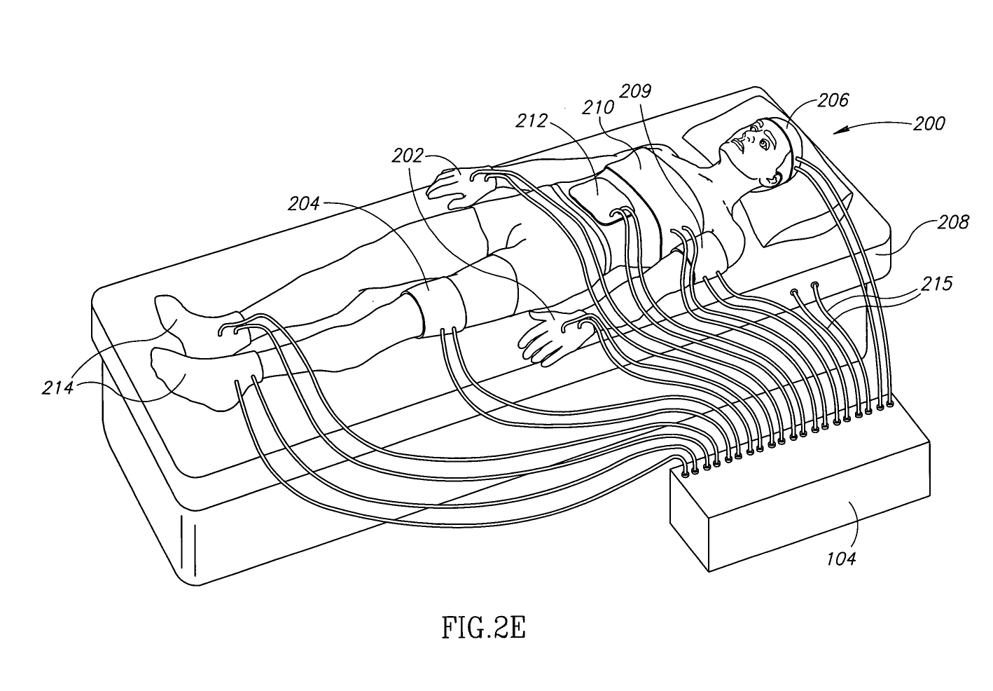

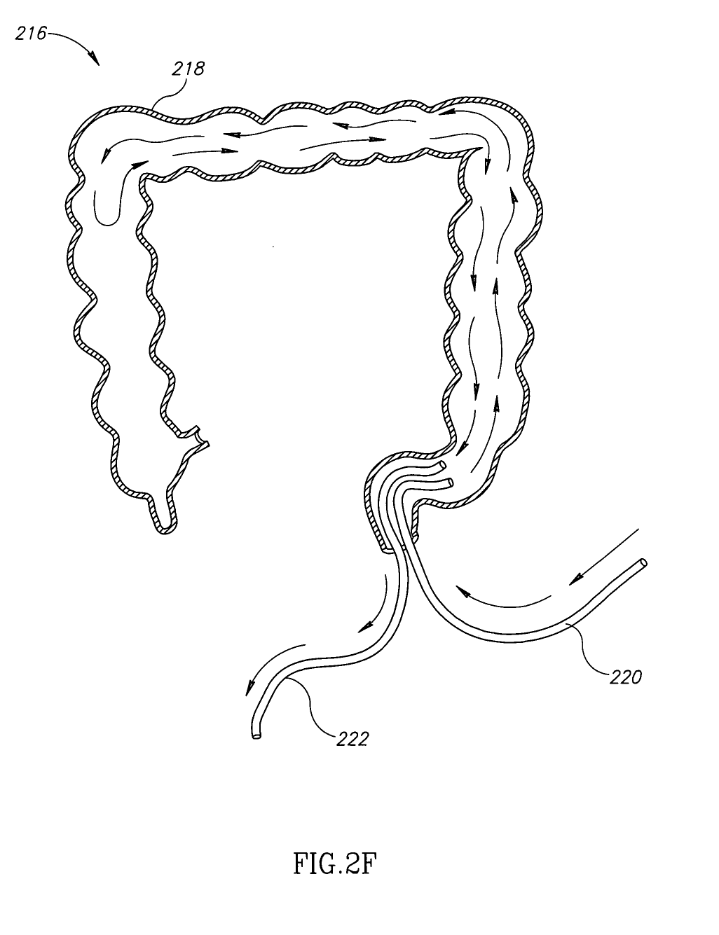

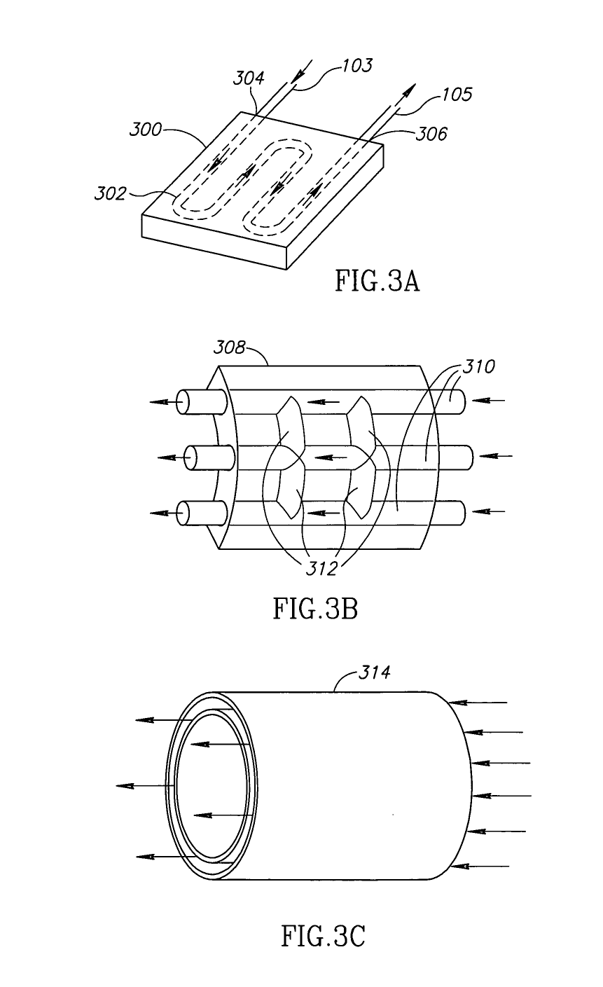

In another embodiment of the invention, there is no circulating fluid in the cooling patch, but the refrigeration unit is combined with the cooling patch. In such embodiments, the refrigeration unit is optionally thin and flexible, for example it is a Peltier unit as shown below in FIG. 4A, so it is not awkward to wear against the body. Optionally, the refrigeration unit is not worn under clothing, allowing heat generated by the unit to be lost effectively, for example by convection in the ambient air, without reheating the body. Optionally, even if the refrigeration unit is worn under clothing, a heat transfer system, for example using water coils, is located outside the clothing.

In another embodiment of the invention, a pack of a cooling material is held against the body, and replaced when it becomes relatively warm. Optionally, the material has a relatively high heat of fusion, and a melting point at a temperature that would feel cool but not cold against the skin, for example about 15 degrees Celsius, or about 18 degrees Celsius. Using a material with these properties has the potential advantage that the material will remain at the melting point temperature for a relatively long period, as it removes heat from the body. As noted above, in some embodiments of the invention such a cooling material is used in refrigeration unit 104, to remove heat from the cooling fluid. Optionally, the cooling material is used instead of or in addition to using electric power to run the refrigeration unit. When used for that purpose, it may be advantageous for the cooling material to have a somewhat lower melting point than a temperature in a range at which the cooling fluid is maintained when it is cooling the skin.

Controller 106 optionally controls refrigeration unit 104, turning it on or off as needed for example, and optionally controls the rate of pumping of fluid through cooling patch 102, or turns the pumping on and off. Controller 106 optionally does this in response to input from one or more sensors, in a closed feedback loop. The sensors include, for example, one or more of a sensor 108 which measures the intensity of shivering by the subject; a sensor 110 which measures blood flow, for example in skin adjacent to cooling patch 102; and a sensor 112 which measures temperature, for example one or more of the temperature of the skin adjacent to the cooling patch, the inlet temperature of the fluid going into the cooling patch, and the outlet temperature of the fluid. As will be described in more detail below, one or more feedback loops optionally allow the cooling system to operate in a regime where blood flow is not reduced by vasoconstriction, and/or where the cooling patch does not cause discomfort.

In some embodiments of the invention, there is a sensor 132 which measures base metabolic rate, for example by measuring oxygen consumption while the subject is resting, and the cooling system removes heat from the body at a great enough rate or average rate, over a long enough period of time, to raise the subject's base metabolic rate over a long term, as measured by the sensor. Optionally, the subject's base metabolism goes up by at least 10%, or at least 20%, or at least 40%, and the increase lasts for at least one day, or at least one week, or at least one month. Optionally, the base metabolic rate is measured at times when the body is not being cooled, in order to verify the long term nature of the increase in base metabolism induced by the cooling. Sensor 132 works, for example, by measuring the concentration of oxygen and/or the concentration of carbon dioxide, in a face mask worn by the subject while inhaling and exhaling. The raw data is optionally analyzed by controller 106 to find the rate of oxygen consumption, for example by multiplying the breathing rate by the change in oxygen volume or the change in carbon dioxide volume per breath.

FIG. 1 also schematically shows parts of the body. Cooling patch 102 is optionally located directly against skin 122, or is located close to the skin, separated from it by a thin layer of cloth for example, or another material. The cross-section of skin 122 is not drawn to scale in FIG. 1, but is made thicker than it would usually be in proportion to components of cooling system 100, in order to show parts of the skin more clearly. Optionally, the direct effect of cooling patch 102 is primarily to draw heat out of capillaries 124 in the skin, above a layer 126 of subcutaneous fat. Initially, the body may respond by vasodilatation of capillaries 128 which connect capillaries 124 to small blood vessels 130 under subcutaneous fat layer 126, increasing blood flow to the skin, to maintain its temperature. But prolonged cooling may cause vasoconstriction leading to a decrease of blood flow in capillaries 128, in order to conserve heat deeper in the body. In either case, the body's thermal regulation system responds to the decrease in temperature of the blood by increasing small rapid contractions in the muscles, which generates heat, and, if the cooling continues, by causing shivering, which is a larger amplitude lower frequency periodic contraction of the muscles, which generates more heat. These contractions of the muscles consume glucose, lowering the glucose level in the blood. The increased energy utilization may cause mobilization of fat. The result is a decrease in the mass of stored body fat, if the subject does not increase food intake to supply the calories being consumed by the contractions of the muscles. The cooling may also cause the heart to pump harder, to maintain the temperature in the core of the body, at least, and to keep the muscles supplied with glucose and oxygen. The increased heart rate may have health benefits as well, similar to the benefits of aerobic exercise for example, apart from the health benefits of the decrease in the mass of stored fat.

Shivering can consume 2650 kilocalories in 24 hours, somewhat more than the number of kilocalories metabolized by an average man who does not do much exercise. Although the cooling system would probably not be used 24 hours a day, and might not cool the subject enough to cause shivering, this number shows that, if it is used several hours a day, the cooling system can potentially cause weight loss at a rate comparable to that produced by a restricted diet, or by strenuous exercise. Optionally, the cooling system can remove at least 10 kilocalories of heat from the body in one hour, or at least 20 kilocalories or at least 50 kilocalories or at least 100 kilocalories in one hour.

Using feedback and/or other methods (to be described below), to reduce or avoid vasoconstriction, has the potential advantage that it can increase metabolism more effectively than uncontrolled exposure to cold. These methods of cooling may not need to be used for a very long time to be effective. Using feedback and/or other methods (to be described below) to avoid discomfort, on the other hand, has the potential advantage that a subject may tolerate the use of cooling system 100 for a longer period of time than if uncontrolled cooling were used. For either or both these reasons, patients may continue the use of cooling system 100 for a long enough time to lose significant weight. Furthermore, the same methods that avoid discomfort to the patient also have the potential advantage that they may prevent damage to the body (e.g. frostbite, or reduction in the core temperature of the body). This may allow cooling system 100 to be used safely at home, without supervision of a physician, as might be necessary if cooling were accomplished by putting the subject in a cold room, making cooling system 100 less expensive to use.

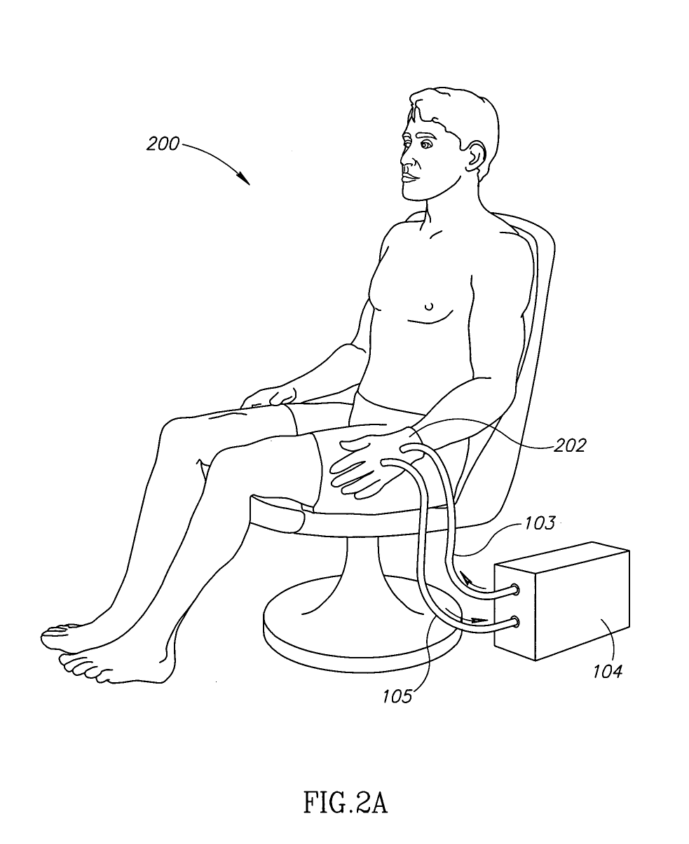

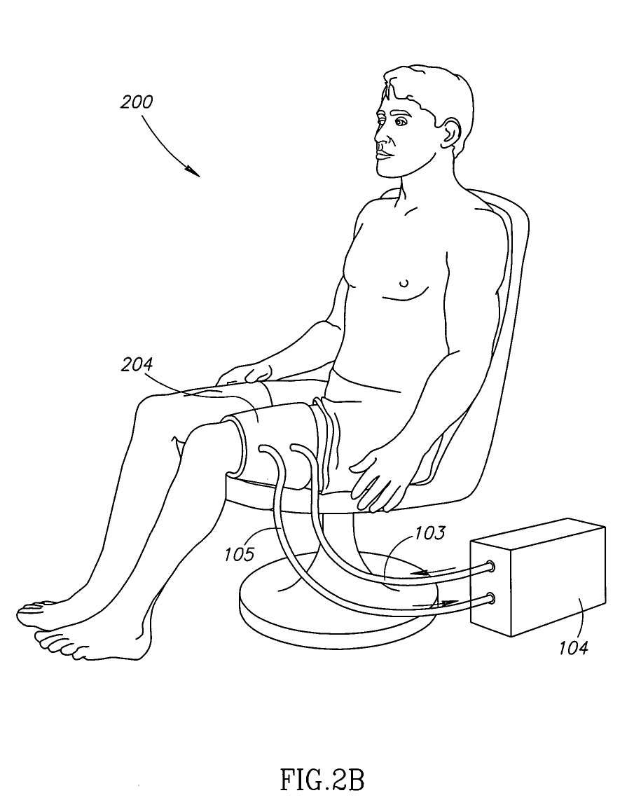

Cooling patches similar to cooling patch 102 are optionally sized and shaped to be worn on a variety of different parts of the body. FIG. 2A shows a subject 200 wearing a cooling patch 202 in the form of a glove on the hand. A cooling fluid is cooled in refrigeration unit 104, and flows through inlet tube 103 to cooling patch 202, where it circulates, absorbing heat from the subject's body, before returning through outlet tube 105 to refrigeration unit 104 where it is cooled again before returning to cooling patch 202. If subject 200 wears cooling patch 202 on his left hand, and works primarily with his right hand, then he may be able to wear cooling patch 202 during working hours, without it interfering very much with his work.

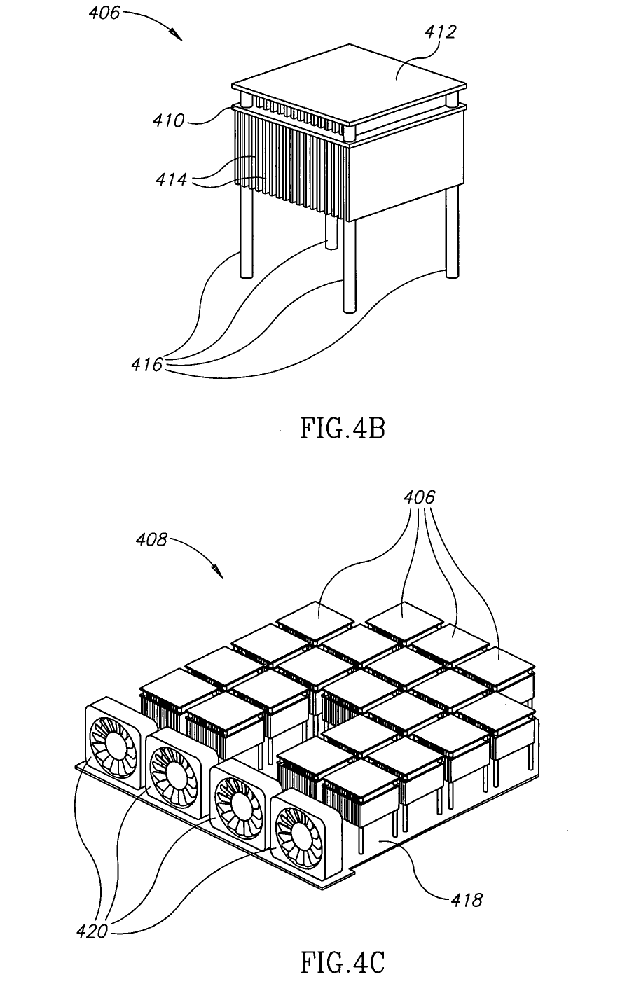

Optionally, the contact between glove 202 and the subject's hand is improved by using a thermally conductive liquid such as a gel or a cream, and/or by making the glove elastic or otherwise pressing the glove against the subject's skin, and/or by applying a partial vacuum to the glove, as described below in connection with FIG. 6. Any of these methods, or any combination of these methods, is optionally used for improving thermal contact between any of the other types of cooling elements or cooling patches described herein, and the subject.

Optionally, glove 202, or any form of cooling patch used, includes a digital or graphical display, not shown in FIG. 2A, which displays one or more of the inlet temperature, the outlet temperature, the difference between the inlet and outlet temperatures, the skin temperature, the elapsed time, the flow rate, and the cumulative heat loss. Displaying the cumulative heat loss in kilocalories has the potential advantage that it can encourage the subject to continue using the cooling system. Displaying the other data has the potential advantage that it can indicate if the system is functioning properly. In some embodiments of the invention, data of physiological interest is measured and displayed, for example the heart rate, the respiration rate, glucose and lipid levels in the blood, and metabolic rates of muscle tissue and fat tissue. Displaying this data has the potential advantage that it can help the subject, or supervising medical personnel, assess the effectiveness and safety of the procedure, and make adjustments to improve effectiveness and/or safety, if the data is suitably analyzed.

FIG. 2B shows subject 200 wearing a cooling patch 204 in the form of a band around his thigh. Cooling patch 204 is optionally worn under outer clothing, not shown in FIG. 2B, with inlet tube 103 and outlet tube 105 passing through a hole in the outer clothing, for example a hole concealed in a pocket, and/or passing around the outer clothing. Like cooling patch 202, cooling patch 204 may optionally be worn during working hours without interfering very much with normal work, if the subject works while seated.

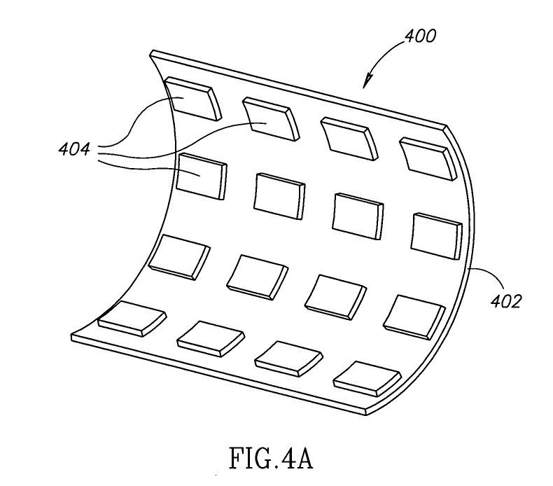

In some embodiments of the invention, a cooling system is used in or on a piece of furniture that a subject sits on, or lies on, or is otherwise in contact with. For example, FIG. 2C shows subject 200 seated on a chair 205, with a cooling system built into it. Inlet tube 103 and outlet tube 105 carry circulating cooling fluid between the chair and refrigeration unit 104. Alternatively, the cooling system is built into a cushion or seat cover placed on the chair. Whether built into the chair, or incorporated into a separate cushion or cover, the cooling system may be used on the seat, the back of the chair, and/or the arm rests. In the case of a recliner, the cooling system may be used additionally or alternatively on leg rests, a head rest, and/or a neck rest. Chair 205 may be used while the subject is working or engaged in other activities. Optionally, any pump used to circulate fluid is relatively quiet, so it will not disturb the subject, and means are used, for example as described below in the description of FIG. 5, to keep the subject comfortable, so he or she will not be distracted. In some embodiments of the invention, a chair or another piece of furniture with a cooling system can adapt its shape to the body of the subject, in order to increase the area of contact.

Optionally, the cooling system is built into modular covers, that can be used, for example, to cover one or more of the seat of a chair, the back of the chair, and the arm rests. Tests have shown that, for a cooling system built into a chair cover that includes the seat and the back of a chair, the subject can lose as much as 35 or 40 kcal/hour. In some but not all subjects, this cooling rate can be achieved without taking any special measures to prevent vasoconstriction, such as those measures described below in the description of FIGS. 5-13.

Optionally, the covers come in a variety of different sizes and/or shapes, to fit a variety of chairs or other pieces of furniture. Optionally, the covers come in a variety of designs, for example in different colors and made from different materials, so they look attractive to different customers and/or in different environments. In some embodiments of the invention, the cooling function of the cover, or of a piece of furniture with a built-in cooling system, is not evident to a casual observer, so the user can use it around other people, at home or at work, without other people knowing that the user is trying to lose weight. In other embodiments of the invention, the cooling system is conspicuous, for the benefit of users who want it to be known that they are using the system to lose weight.

FIG. 2D shows a subject 201 wearing a necklace 203, comprising a cooling system. Optionally, as in the drawing, the cooling system uses Peltier refrigerating units, as described below, which only need a source of electric power, obtained, for example, from an AC-to-DC converter 207 plugged into a wall outlet. Heat is dissipated into the air from the outer surface of the necklace, with relatively little of the heat going back into the subject's body, while the cold surfaces of the cooling system are directly against, or close to, the subject's skin. Alternatively, the cooling system may use circulating cooling fluid, as in FIGS. 2A-2C, in which case the heat may be dissipated at some distance from the subject, by a separate refrigeration unit.

Optionally, the jewelry is in contact with at least 10 square centimeters of the subject's skin, or at least 30 square centimeters, or at least 100 square centimeters, or at least 300 square centimeters, or at least 1000 square centimeters. Optionally, the volume of the jewelry is less than 1 mm times the contact area with the subject's skin, or between 1 and 3 mm times the contract area, or between 3 and 10 mm times the contract area, or more than 10 mm times the contact area. Optionally, the density of the jewelry is less than 1 gram per milliliter, or between 1 and 3 grams per milliliter, or more than 3 grams per milliliter, or more than 7 grams per milliliter.

Cooling systems such as those shown in FIGS. 2A-2D may also be built into, or used together with, other items of clothing, and furniture such as beds, toilet seats, sofas, recliners and lounge chairs, and seats in vehicles such as car seats, and seats on planes, trains, buses, and ships. They may be built into other items of jewelry, such as bracelets, brooches, earrings and rings. Cooling systems may also be built into exercise equipment, such as exercise bicycles, or weights, and used while exercising. They may be built into playground equipment, such as swings and see-saws, and amusement park rides, as well as into child-sized chairs for use at home or school. These uses may be particularly advantageous, since obesity has become more common among children in recent years.

When incorporated into a chair, the cooling system may be used in theatres, concert halls, sports stadiums, doctor's waiting rooms, dentists' offices, barbershops, bars, and restaurants. The controller optionally keeps track of how many calories of heat were removed from the customer's body, and the bill optionally includes an account of the number of calories removed, as well as, in the case of a bar or restaurant, an account of the number of calories consumed in the meal, and the net number of calories consumed, whether positive or negative. Similarly, when incorporated into a sofa or chair used to watch television, the controller optionally keeps track of the number of calories of heat removed from the subject's body, and displays the number on the TV screen. The sofa or chair optionally also includes a sensor which detects when the subject sits down and gets up, based for example on the subject's weight, and/or on the thermal load on the cooling system, and only counts cooling that is done when the subject is using the sofa or chair.

FIG. 2E shows subject 200 wearing cooling patches in a variety of forms, while sleeping on bed 208. The cooling patches shown in FIG. 2C include gloves 202, a leg band 204 worn around the thigh or lower leg, a hat or head band 206, an arm band 209 worn around the upper or lower arm, a chest covering or band 210, a “belly pack” 212 worn against the stomach or abdomen, and socks 214. These cooling patches are optionally worn singly, or in any combination. FIG. 2E shows all of the cooling patches receiving fluid from a single refrigeration unit 104, but optionally two or more refrigeration units are used. Optionally, bed 208 has its own cooling system, or a cooling system built into a mattress, pad, or blanket. Optionally, as shown in FIG. 2E, the cooling system of bed 208 also receives circulating cooling fluid from refrigeration unit 104, through tubes 215, or from a separate refrigeration unit.

Although all or most of the cooling patches may also be worn while awake, some of them may be more convenient to wear while sleeping, because they may interfere with activities done while awake, and/or because the subject may feel awkward wearing them in public. The cooling patches may optionally be worn while the subject is alone, and removed when the subject is with other people. If refrigeration unit 104 is not easily portable, for example if it weighs more than a few kilograms, that may make it more difficult to wear the cooling patches while walking around, for example. If the cooling patches are worn while sleeping, then it is potentially advantageous if the tubes connecting refrigeration unit 104 to the cooling units are long enough and flexible enough to allow the subject to turn and move normally or relatively normally in his sleep. Alternatively, there are one or more refrigeration units 104 that are small and lightweight, and optionally built into the cooling patches, for example using Peltier units, so that they do not interfere very much with the subject's movements when sleeping. These small and lightweight units are optionally supplied with electric power by an electric cord plugged into an electric outlet, with the cord optionally long enough to allow the subject to move while sleeping. If refrigerating unit 104 is combined with the cooling patch, then the cooling patch is preferably not worn under the clothing or under a blanket, but is exposed to the air so that it can lose heat easily to the air. In this case, hat 206 or glove 202, for example, may be suitable.

The choice of what time of day the cooling patches are worn may also depend on when the subject eats meals. For some subjects, cooling may be more effective for weight loss if it is done after meals, when the subject has a full stomach. For example, if cooling is done just before meals, then the subject may tend to eat more to compensate for the loss of calories, while this may not be true if cooling is done with a full stomach. For other subjects, cooling may be more effective if it is done before or between meals. For example, after meals there may be less peripheral blood flow, since more blood is being used by the stomach, and it may be more difficult to lose heat than before or between meals. There may also be differences in the amount of heat generated by the body, before and after meals.

Cooling patches which are worn under the outer clothing, for example belly pack 212, or leg band 204, preferably are used with a separate refrigerating unit 104, or at least a separate heat loss unit, located outside the clothing optionally some distance away, so it can lose heat, for example to the air by free or forced convection, without heating the body significantly. Optionally, refrigerating unit 104, and tubes connecting it to the cooling patch, can be temporarily disconnected from the cooling patch, and the tubes and cooling patch can be sealed off. The cooling patch then remains in place hidden by the clothing, if the subject needs to interact with other people and does not want to be seen carrying around refrigerating unit 104.

In some embodiments of the invention, cooling elements are inserted into the body, for example into the mouth, the vagina, the rectum, or other bodily orifices, such as the nose, or the ear canals. The cooling elements may serve also serve a function other than cooling and weight loss. FIG. 2F, for example, shows a colonic irrigation system 216, for cleaning out colon 218. Such systems are commonly used by some people for their supposed health benefits, and the marginal cost of also using it for cooling may be relatively small. A fluid, for example water with an appropriate level of dissolved electrolytes to prevent loss of ions from the body, is introduced into the colon through an inlet tube 220, and pumped out through an outlet tube 222. If the water in inlet tube 220 is sufficiently cooler than body temperature, and if the process continues at a sufficiently great flow rate for a long enough time, substantial heat may be removed from the body, which may lead to an increased metabolism to maintain the core body temperature. Cooling system 216 optionally does not use a closed cycle of fluid circulation, as in 100 system 100 in FIG. 1, but an open cycle, with the waste fluid disposed of after passing through outlet tube 222. Alternatively or additionally, cooling fluid is circulated in the colon in a closed tube, and optionally the same fluid is circulated repeatedly, and cooled outside the body, for example in a refrigeration unit as in FIG. 1.

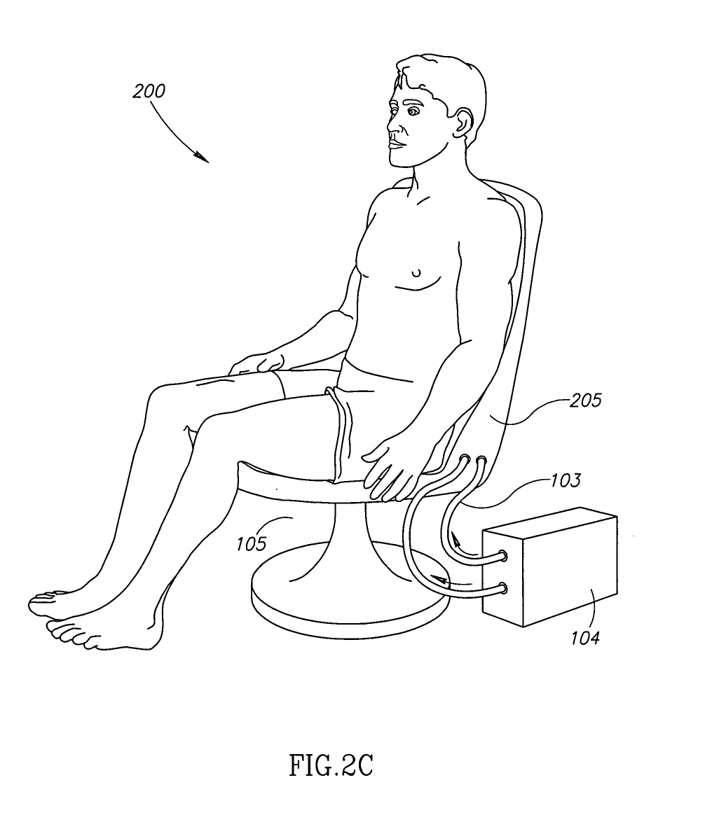

FIG. 3A shows a tube 302 going back and forth in cooling patch 300. Water enters cooling patch 300 at inlet 304, and leaves cooling patch 300 at outlet 306. Although a single tube 302 is shown in FIG. 3A, optionally there are two or more tubes which run in parallel, at least over part of their length. This is the case with tubes 310 in cooling patch 308, shown in FIG. 3B. Optionally, there are one or more cross-tubes 312 connecting the parallel tubes. There are several tradeoffs that govern the choice of tube diameter, tube spacing, number of tubes, water pressure, and flow rate, for a cooling patch of a given area, designed to be used on a given part of the body. These parameters optionally have different values for at least some of the different tubes.

One tradeoff involves the total length of the tube, or of each tube if there are two or more tubes in parallel. If a tube is too short, and the flow rate is too great, then the water will not increase in temperature very much while it is going through the tube, and much of the power used to pump the water through the tube will be wasted. On the other hand, if the tube is too long or the flow rate is too low, then the water will reach thermal equilibrium with the skin and the outside air before it has gone through most of the length of the tube, and after that the water will not do much cooling, so again much of the pumping power will be wasted. Optimally, the water will rise in temperature by a significant fraction of the temperature difference between the initial temperature and the equilibrium temperature, in the course of going through the length of the tube.

A tradeoff also exists for the ratio of tube spacing to tube diameter. If adjacent parts of the tube are too far apart compared to their diameter, then they will only cool a small fraction of the area of the skin under the cooling patch. But if the tubes are too close together, then one part of the tube may cool an adjacent part of the tube, rather than cooling the skin, making the cooling system less efficient.

For a given ratio of tube diameter to tube spacing, using a smaller diameter tube will make the cooling patch flatter, making it easier to fit the patch under clothing, and/or to conform the patch to the curved surface of the body. But if the tube is too small in diameter, and/or there are too few tubes in parallel, then it will take more pressure, and hence more power, to pump a given flow rate of water through the tube. This is especially true if the cross-sectional area of the tube or tubes is so small that the flow is turbulent at the desired flow rate. If the pumping power is a significant fraction of the refrigerating power, then the cooling system will be using power inefficiently. Furthermore, the power lost to drag of the water against the surface of the tubes, whether the flow is laminar or turbulent, will heat the water, making the water less effective at cooling the skin. This will set a minimum total cross-sectional area of the tube or tubes, to get efficient operation at a given flow rate.

A potential advantage of using a plurality of tubes in parallel, rather than a single tube, is that fluid flow will not be disrupted everywhere in the cooling patch, if the subject presses against the cooling patch, for example inadvertently, and squeezes one tube so that it is partly or completely closed.

The inventors have found that tubes of 2 to 3 mm in diameter work well when water is used as the cooling fluid. Typically, between a few tens and a few hundred milliliters of water are pumped per minute, for a cooling patch comprised in a glove. When uncompressed air is used as the cooling fluid, the inventors have found that tubes of about 1 centimeter in diameter work well. The volume of uncompressed air pumped per minute is generally higher than the typical volume of water pumped per minute, to obtain the same rate of heat flow, because uncompressed air has a much lower heat capacity per volume than water. Using compressed air, or another compressed gas, particularly one with a higher heat capacity than air, has the potential advantage that it has a higher heat capacity per volume than uncompressed air. But using uncompressed air or gas has the potential advantage that there is no need to use a compressor, and using air, compressed or uncompressed, has the potential advantage that there is no need to supply a canister of gas.

The tube or tubes need not have a circular cross-section. A tube of non-circular cross-section may make better use of the available space then two or more smaller parallel tubes of circular cross-section. Optionally, as shown in FIG. 3C, the entire space between two concentric sleeves is used for fluid flow, in the case of cooling patch 314 that is in the form of a band.

FIG. 4A shows a cooling patch 400 in which Peltier refrigerating units 404 are mounted on a thermally conducting backing 402. The Peltier effect pumps heat from the surface of units 404, which is at a lower temperature, in contact with or close to the skin, and transfers the heat to backing 402, at a higher temperature. The heat is lost from the back of backing 402 (i.e. the surface of backing 402 not visible in FIG. 4) to the air, through free or forced convection (using a fan, for example), and, generally to a lesser extent, through radiating infrared. Peltier units 404 are optionally designed and used in a way so that the back of backing 402 is well exposed to the air, and, particularly if free convection is being used, so that the back of backing 402 faces upward, or at least does not face downward. Optionally, cooling patch 400 is designed and used in a way that allows the heat to convect away from cooling patch 400, with little of the heat going back into the body.

The dimensions of cooling patch 400 need not have the ratios shown in FIG. 4A, but are optionally determined by tradeoffs, for example between having a thin, lightweight cooling patch that is comfortable to wear, and having efficient and effective cooling.

FIG. 4B shows a thermoelectric cooling module 406, suitable for use in a cooling patch 408 shown in FIG. 4C, which uses forced convection to remove heat. Cooling module 406 comprises a Peltier element 410, a cold plate 412 in thermal contact with the cold side of Peltier element 410, and a heat sink 414, optionally comprising multiple plates arranged parallel to each other, in thermal contact with the hot side of Peltier element 410. Module 406 optionally also includes mounting posts 416. In FIG. 4C, a plurality of cooling modules 406 are mounted in an array on a base 418, using posts 416. Optionally, the array is not completely filled in with cooling modules, but the cooling modules are located at positions where they will be most effective at removing heat from the body, for example opposite the parts of the thighs and buttocks that are in the best thermal contact with the cooling patch, and/or are least subject to vasoconstriction. Keeping other parts of the array free of cooling modules has the potential advantage that it may be easier to remove heat from the cooling modules that are present. The posts provide for air space between heat sinks 414 and base plate 418. A row of fans 420 causes air to flow past the heat sinks of modules 406, removing heat from the heat sinks. The air optionally flows through the spaces between the plates, which are optionally oriented with their surfaces parallel to the direction of air flow. The air also optionally flows in the space between the heat sinks and base 418. Optionally, the fans blow air past the heat sinks; alternatively, the fans blow air away from the array of cooling modules, thereby drawing air past the heat sinks.

Cooling patch 408 may be used, for example, in any of the devices described above in connection with FIGS. 2A-2E, instead of or in addition to using circulating fluid for cooling. FIG. 4D shows cooling patch 408 used as part of a chair, attached to the top of a modified wooden chair seat 422, for example by bolts. The fans in front of cooling patch 408 (labeled 420 in FIG. 4C) optionally draw in air from inlet vents 424 in the front of seat 422, and blow the air past the heat sinks (labeled 414 in FIG. 4B) of cooling patch 408. The heated air is then optionally guided down through exhaust vents 426, optionally located on the sides of seat 422, as shown in FIG. 4D, and/or in the back of seat 422, where they are hidden from view in FIG. 4D. Optionally there is a cover 428 over cooling patch 408, which forces the heated air down through exhaust vents 426. This arrangement has the potential advantage that the heated air is directed away from a subject, not shown, seated on top of cooling patch 408, so that the heated air does not heat the subject. Cover 428 is drawn with dashed lines, and shown as transparent in FIG. 4D, so that cooling patch 408 will be visible, but cover 428 need not be transparent. Optionally, the top surface of cover 428 is in good thermal contact with the cold plates (labeled 412 in FIG. 4B) on the top of cooling patch 408, and optionally the top surface of cover 428 is itself a good thermal conductor, so that the cold plates can efficiently cool the subject. In some embodiments of the invention, cover 428 is open on top, or is not present at all, so that the subject can sit directly on the cold plates of cooling patch 408.

In some embodiments of the invention, the heated air may be intentionally directed to heat a different part of the subject's body than is being cooled, as described below.

Optionally, whether cooling patch 408 is used in an article of clothing or jewelry, or a piece of furniture, base 418 is made of a flexible material, allowing the cold plates of the cooling modules to conform to the subject's skin over an extended area, even if the small individual cooling plates of the cooling modules are rigid. Alternatively, as shown in FIG. 4D, base 418 of cooling patch 408 is rigid and flat.

Optionally, the lower surface of 418 comprises a compressible of flexible material, such as foam rubber, which can mold itself to fit a curved chair surface, or the lower surface of base 418 is rigid and curved to fit a particular chair surface, such as seat 422 in FIG. 4D. Alternatively, base 318 is rigid but thin, and both the top and bottom surfaces of base 418 are curved like the surface of seat 422. In this case, if each of the cooling modules in cooling patch 408 has the same height, then the top surface of cooling patch 408 would have the same curvature as seat 422, which has the potential advantage that it might conform better to the contours of the subject's body, possibly providing a more comfortable surface to sit on, and/or better thermal contact between the subject and cooling patch 408.

In some embodiments of the invention, the top of cooling patch 408, and optionally the top of base 318, is rigidly curved, but with a different shape than seat 422. The curvature of the top of cooling patch 408 can be designed, for example, to provide good thermal contact with particular parts of the subject's thighs or buttocks, which have been found to be especially effective for cooling, and/or to provide better comfort to the subject during cooling.

Optionally, if seat 422 comes from an existing chair, then the legs of the chair are shortened by an amount approximately equal to the height of cooling patch 408, so that cooling patch 408 is as comfortable to sit on as the original chair.

In some embodiments of the invention, instead of using seat 422 which is irreversibly modified by having vents cut into it, cooling patch 408 is attached in a reversible way, for example by straps, to an existing chair seat which is not permanently modified.

In some embodiments of the invention, a cooling patch uses a combination of direct Peltier cooling of the skin, and cooling the skin by circulation of water or another fluid cooled remotely in a refrigerating unit. The refrigerating unit uses, for example, a compression-expansion cycle, or Peltier cooling, or evaporative cooling, or any other method of cooling known in the art, or any combination of these methods. In some embodiments of the invention, an array of Peltier units cools the skin, as in FIG. 4C, while circulating water cools the heat sinks of the Peltier units, instead of or in addition to air blown by fans.

In some embodiments of the invention, cooling is concentrated locally on certain blood vessels that are near the surface of the skin, and are less subject to vasoconstriction, or less subject to locally induced vasoconstriction, than other blood vessels. For example, veins may be less subject to locally induced vasoconstriction than arteries. Small or at least narrow Peltier units may be especially effective for such local cooling, but cooling by circulation of a fluid may also be used. Optionally, a cooling patch has tubes running parallel to veins near the surface of the skin, and a cooling fluid runs through the tubes which preferentially cools the veins, while the rest of the skin under the cooling patch optionally is thermally insulated from the tubes, to reduce or prevent a local vasoconstrictive response of the arteries.

In some embodiments of the invention, blood is made to flow from a vein through a tube outside the body, where it is cooled, and then returned to the vein, or another vein. The cooling of the blood outside the body is optionally passive, relying on the lower ambient temperature outside the body. Additionally or alternatively, the cooling is active, using any of the cooling methods described above. This method may be especially useful for dialysis patients, who are already having blood removed from and returned to their bodies in the course of dialysis treatment, and who often suffer from obesity.

Various methods are optionally used to reduce or prevent discomfort caused by the cooling device. Some of these methods may prevent damage to tissue from excessive cold. In some embodiments of the invention, cooling is done at a fixed duty cycle. The duty cycle is optionally determined in advance, or on an on-going basis, for example, by tests which find what duty cycle will avoid discomfort or tissue damage. In other embodiments of the invention, a feedback loop is used to turn the cooling on and off.

FIG. 5, for example, shows a flowchart 500 for a feedback loop, in which cooling is controlled so as not to cause too low a skin temperature, too low a blood flow rate, or too much shivering. At 504, the skin is cooled, for example by turning on the refrigerating unit, or turning on a pump which pumps cold water through the cooling patch. At 506, the skin temperature adjacent to the cooling patch is optionally measured. Additionally or alternatively, the water temperature is measured at one or more locations in the cooling patch, for example at the inlet, or halfway between the inlet and the outlet. Additionally or alternatively, one or more of these measured temperatures, optionally together with the flow rate of the water, is used to estimate an effective temperature of the subject's temperature-sensitive and/or pain-sensitive nerves in the skin, which will affect the discomfort felt by the subject. Additionally or alternatively, the subject's core temperature is measured or estimated, for example with a rectal or tympanic thermometer. At 508, a shivering sensor, for example an accelerometer sensitive to an appropriate frequency range around a few Hz, is optionally used to measure the degree of shivering caused by the cooling. At 510, blood flow is optionally measured, in the capillaries adjacent to the cooling patch, for example by using photoplethysmography, or any other method known in the art for measuring peripheral blood flow. The measurements made at 506, 508, and 510 need not all be made, need not be made in the order shown, and some or all of them may be made simultaneously.

Tests are made at 512, 514, and 516, comparing each of the measured data to threshold values. At 512, the measured temperature, or the estimated effective temperature of the nerves, or any other combination of two or more measured temperatures, is optionally compared to a threshold temperature, for example a temperature below which the subject is likely to become uncomfortable. If the measured temperature is too low, then the cooling is stopped at 518, for example by turning off the refrigerating unit or turning off the pump. The threshold temperature is optionally about 10 degrees Celsius, or about 12 degrees, or about 14 degrees, or about 16 degrees, or about 18 degrees, or about 20 degrees, or about 22 degrees, or about 25 degrees, or about 30 degrees, or any lower, higher, or intermediate temperature.

If the measured temperature is not too low, then the measured amplitude of shivering is optionally compared to a threshold amplitude at 514, for example an amplitude of shivering that indicates that the subject is likely to be uncomfortably cold. If the measured shivering amplitude is too great, then the cooling is stopped at 518.

If the measured shivering is not too great, then the blood flow rate is optionally compared to a threshold blood flow at 516. The threshold blood flow, for example, may be a blood flow that is substantially less than the normal blood flow rate in that part of the skin, for example less than 50% of the normal blood flow rate. A blood flow rate lower than this indicates that significant vasoconstriction is occurring, as a result of the local cooling, and further cooling of the skin in that region will be less effective at removing heat from the body. If the blood flow is too low, then cooling is stopped at 518. If the blood flow is not too low, then cooling is continued, at 504. Tests done by the inventor have shown that there is a wide variation, among different people, in how much they can be cooled before significant vasoconstriction occurs.

Optionally, the condition for stopping the cooling at 518 is more complicated than passing independent tests at 512, 514, and 516. For example, the cooling is stopped at 518 if a function involving two or more of temperature, shivering amplitude, and blood flow exceeds a threshold.

Optionally one or more of the thresholds, or other condition for stopping the cooling at 518, is set by the subject at a desired value. The subject may take into account a desired tradeoff between greater discomfort and losing weight more quickly, and/or a personal degree of tolerance or lack of tolerance for cold, in setting the stop condition. Preferably, the subject is not permitted to set a threshold at a level that would be dangerous, for example at a level that would allow his core temperature to fall dangerously low. In some embodiments of the invention, instead of or in addition to being able to set thresholds or conditions for stopping the cooling, the subject can directly stop the cooling, for example if he feels uncomfortable, and resume the cooling later. Optionally, in embodiments where there is a mechanism for heating the subject as well as for cooling, as described below, the subject can start the heating mechanism, in addition to stopping the cooling, if he feels too uncomfortable.

Optionally, there is an emergency shut off mechanism, which shuts off the cooling if the system malfunctions, to avoid unsafe operation. For example, the emergency shut-off mechanism measures one or more of core body temperature, skin temperature adjacent to the cooling system, and heart rate, and shuts off the system if any of these values, or the combination of these values, falls outside a safe range, as determined, for example, by a look-up table. Optionally, the sensors and the control system used by the emergency shut-off mechanism are arranged so that they will be fail-safe, and if a sensor malfunctions, or the power to the sensor is cut off, then the sensor will indicate an unsafe value, and if the control system malfunctions or loses power, then the cooling system will shut off.

If cooling is stopped at 518, then the measurements of temperature, shivering and blood flow are optionally repeated at 506, 508 and 510, until the temperature is high enough, the shivering is low enough, and the blood flow rate is great enough, after which cooling is started again at 504.

The tests of temperature, shivering, and blood flow need not be performed in the order shown in FIG. 5, and all of these tests need not be done. The measurements of temperature, shivering, and blood flow need not all be made before performing any of the tests. Optionally, each measurement is made instead just before performing the corresponding test.

In some embodiments of the invention, if the measured blood flow is too low, then, instead of or in addition to stopping the cooling, other actions are performed to increase blood flow, for example mechanically stimulating the skin by massaging or applying vibrations to the skin adjacent to the cooling patch. FIG. 6 shows a cooling glove 600, for example, with one or more moving elements 602 inside, which are in direct or indirect contact with skin 604. Vibration, rubbing, or other motion of elements 602 against the skin may cause vasodilation, increasing blood flow, and allowing cooling glove 600 to cool the skin more effectively.

In some embodiments of the invention, the cooling system is built into a chair, or into a seat cover placed on a chair, similar to the system shown in FIG. 2C, and the chair includes a massage mechanism. The massage mechanism is optionally used to increase blood flow during cooling. Additionally or alternatively, the massage mechanism is used to give the user a massage when desired by the user, such as would be done with an ordinary massage chair, whether or not the cooling system is being used, and whether or not the user's blood flow rate needs to be increased to maintain the cooling rate. Optionally, elements of the cooling system also function as elements of the massage mechanism, for example there are Peltier cooling units which vibrate to provide a massage.

M. Bovenzi, C. J. Lindsell, and M. J. Griffin, Occupational Environmental Medicine 57:422-430 (June 2000), the disclosure of which is incorporated herein by reference, describes the use of vibration to cause vasodilation, and lists examples of effective amplitudes and frequencies, for example 5.5 M/s² at 16 Hz, and 88 m/s² at 250 Hz. WO 2004/071570 to Horzewski et al, cited above, also describes the use of vibrations (sound waves) at auditory frequencies to cause vasodilation.

Optionally, glove 600 has a vacuum hose 606, connected to a vacuum pump (not shown), which lowers the pressure inside the glove, creating a partial vacuum. The vacuum can cause vasodilation, as described in the paper by Grahn et al, cited above. In addition, or alternatively, the vacuum helps to keep glove 600, and its cooling and vibrating elements, in better contact with the skin. Such a vacuum hose is optionally used with any of the cooling devices described here, to improve contact with the skin. Optionally, glove 600 makes a fairly air tight seal around the wrist, so that the partial vacuum is not broken by air rushing into the glove.

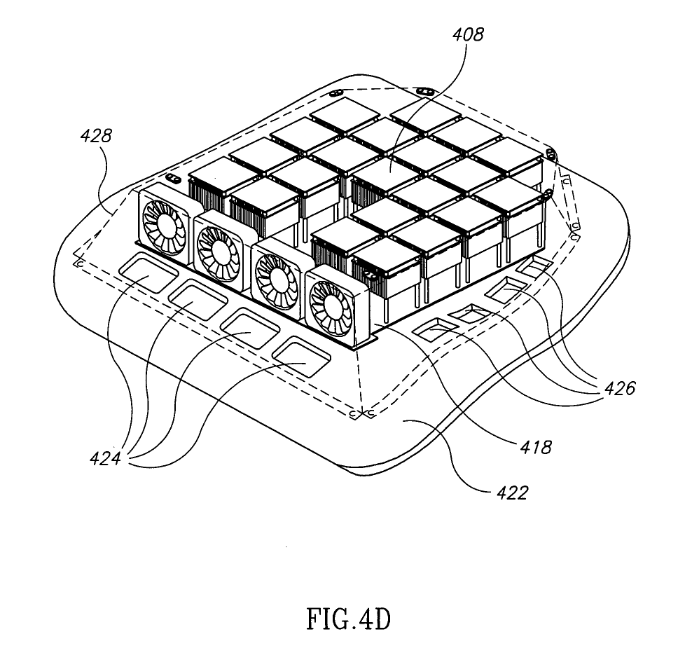

FIG. 7 shows a cooling glove 700, illustrating an alternative method of increasing blood flow, using electrical stimulation. Optionally, this method is used for any other part of the body, and/or is used in conjunction with mechanical stimulation as shown in FIG. 6. A source 704 of electric power, optionally built into glove 700, applies a voltage across electrodes 702, which are in electrical contact with the skin. The voltage stimulates the skin, producing vasodilation and increasing the blood flow. Alternatively or additionally, an electric power source 708, separate from the glove, applies a voltage across electrodes 706, applied to the skin outside the glove, and/or inside the glove with wires extending out of the glove to power source 708. Optionally, electrodes 706 are not located near the glove, but are located at a different part of the body, and produce a systemic vasodilation, rather than local vasodilation, by stimulating a nerve which has a systemic vasodilative effect, for example the vagus nerve. There is a large body of literature on systemic vasodilative effects of stimulating the vagus nerve, for example K. S. Eccles and R. Eccles, Rhinology 20(2), 89-92, June 1982, describes nasal vasodilation due to stimulation of the vagus nerve. This paper is incorporated herein by reference.

The use of electric field stimulation (EFS) induced endothelium dependent vasodilation is described in papers by G. G. Emerson and S. S. Segal, Am. J. Physiol. Heart Circ. Physiol. 280(1):H160-7 (January 2001), and by J. C. Sullivan and C. A. Davison, Cardiovascular Research 50(1):137-44 (April 2001), the disclosures of which are incorporated herein by reference. Electrically induced vasodilation is also described by Tartas et al, cited above.

In some embodiments of the invention, one or more other methods are used to prevent discomfort caused by the cooling device. For example, a topical anesthetic is optionally applied to the skin adjacent to the cooling patch. Adaptation of the body to the cold may occur, if an appropriate inlet temperature is used, so any discomfort may be only temporary. Discomfort may also be reduced by allowing the cooling patch to be in direct contact with the skin only over limited areas, or over several disconnected areas that are separated from each other by some distance. Having a thin layer of insulating material between the cooling patch and the skin may also reduce discomfort, while allowing the body to lose heat at a significant rate. For example, tests have found that an inlet temperature of 11.5 degrees Celsius does not cause discomfort if the water is not in direct contact with the skin. Discomfort may also be avoided by keeping the inlet temperature from getting too low, even without using a feedback loop as described in FIG. 5. For example, tests have shown that water temperatures of about 15 or 18 degrees, even in direct contact with the skin, do not cause discomfort in some people.

Using a pulsed cooling system can also reduce discomfort, with or without feedback, and can prevent vasoconstriction. For example, the cooling device operates for a period of 2 to 5 seconds, before vasoconstriction has time to occur, and is turned off for one or two minutes, enough time for circulation to warm the cooled area. With this mode of operation, the skin temperature may remain high enough to prevent discomfort and vasoconstriction.

In some embodiments of the invention, other actions are performed to produce vasodilation of the capillaries under the skin in the region of the cooling patch, and/or to prevent vasoconstriction. Such actions can increase the effectiveness of the cooling device, and can allow it to operate at a higher temperature, avoiding discomfort, while still removing heat from the body at a substantial rate. Vasoconstriction is prevented or reversed, for example, by using a pulsed cooling system. Vasoconstriction is also optionally prevented by intravenous injection of 5-HT1A receptor agonists, as described by Oostuka and Blessing, cited above. Optionally, 5-HT1A receptor agonists are applied topically, and are absorbed, for example, by iontophoresis. Vasodilation is optionally produced by topical creams applied to the skin, containing calcium channel blockers or alpha blockers, for example benzocaine, nifedipine, and sodium nitrite. Methyl salicylate, menthol, camphor, methyl nicotinate, and/or nitroglycerine are also optionally applied topically to the skin, to cause vasodilation. Vasoconstriction generally does not occur, or occurs only at a greater degree of cooling, when a cooling patch is applied to the abdomen or the thigh, as opposed to peripheral regions of the body such as the hands or feet.

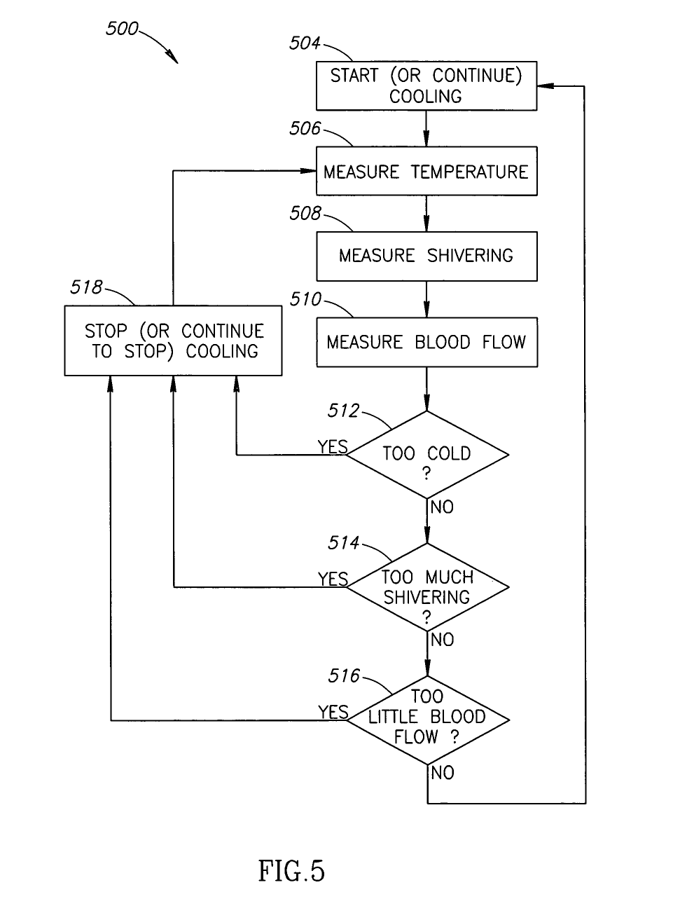

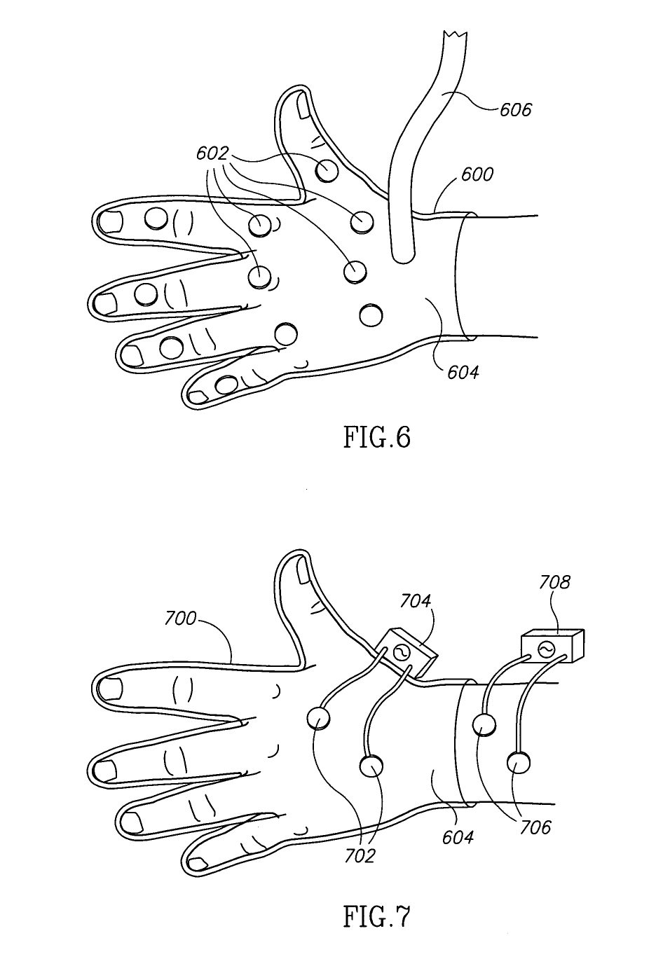

FIG. 8 shows a cooling glove 800, covering a hand with a topical cream 802 applied, which produces vasodilation. Optionally, a cream, or another form of a chemical that produces vasodilation, is exuded from a glove, or from a cooling patch that covers a different part of the body, onto the skin. FIG. 9, for example, shows a cross-section of a cooling patch 902, in contact with skin 904. An outer layer 906 of cooling patch 902 optionally has cooling elements, such as tubes carrying fluid or Peltier units, not shown. An inner layer 908 of cooling patch 902 has pores containing a vasodilation-producing chemical, which is exuded onto skin 904 when cooling patch 902 is pressed against it, compressing the pores. Alternatively or additionally, a cooling patch similar to cooling patch 902 is used to exude a topical anesthetic onto skin 904, to reduce discomfort caused by the cooling, as described above.

In some embodiments of the invention, removal of heat from the body is accomplished entirely by producing vasodilation, rather than cooling the skin. Optionally, vasodilation is increased by heating the skin, and the increased heat loss due to the vasodilation more than compensates for any heat that flows into the body by heating the skin. Optionally, the skin is alternately cooled and heated, back to its original temperature or even higher, to increase vasodilation and increase the heat lost from the body.

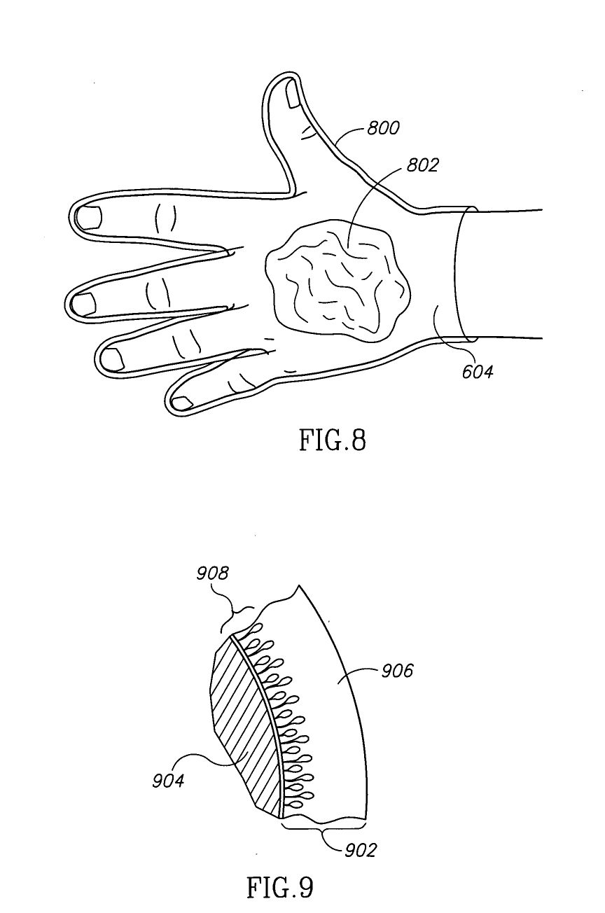

FIG. 10 shows a glove 1000, for example, which is used for alternately heating and cooling the skin of the hand. A similar structure is optionally used for alternately heating and cooling the skin on any other part of the surface of the body. Glove 1000 has four layers. An optional outer layer 1002 thermally insulates the glove from the outside air. A cooling layer 1004, under the outer layer, cools the skin, carrying heat away, for example by fluid flowing through one or more tubes, and/or by Peltier units, neither of which are shown in FIG. 10. Optionally, there is no outer insulating layer 1002. Not having an outer insulating layer may be particularly advantageous if cooling layer 1004 convects and/or radiates heat away to the outside air, for example heat generated by Peltier units, rather than carrying away heat by flowing fluid. A heating layer 1006, optionally under the cooling layer, heats the skin, for example by using heated fluid flowing through tubes, or by generating heat locally, for example electrically, for example using resistors or resistive wire. Alternatively, heating layer 1006 is outside cooling layer 1004.

In some embodiments of the invention, there is a single layer which functions both as a heating and a cooling layer. For example, the layer comprises Peltier units which either heat or cool the skin, depending on the polarity of voltage applied to the Peltier units. Additionally or alternatively, a single tube or set of tubes is used to pump either warm or cool fluid past the skin.

In some embodiments of the invention, the heating function of the system may be used to keep the subject warm, for example when the air temperature is too cold for comfort, even when the system is not being used for weight loss. For example, there is a control mechanism which the subject can use to set a temperature, or a level of heating such as “high,” “medium” or “low,” similar to the control mechanism of an electric blanket. This additional use for the system, with little additional cost in the controls, may make the system attractive to customers who are not interested or are only marginally interested in using it for weight loss, or who do not want people to know they are using it for weight loss.

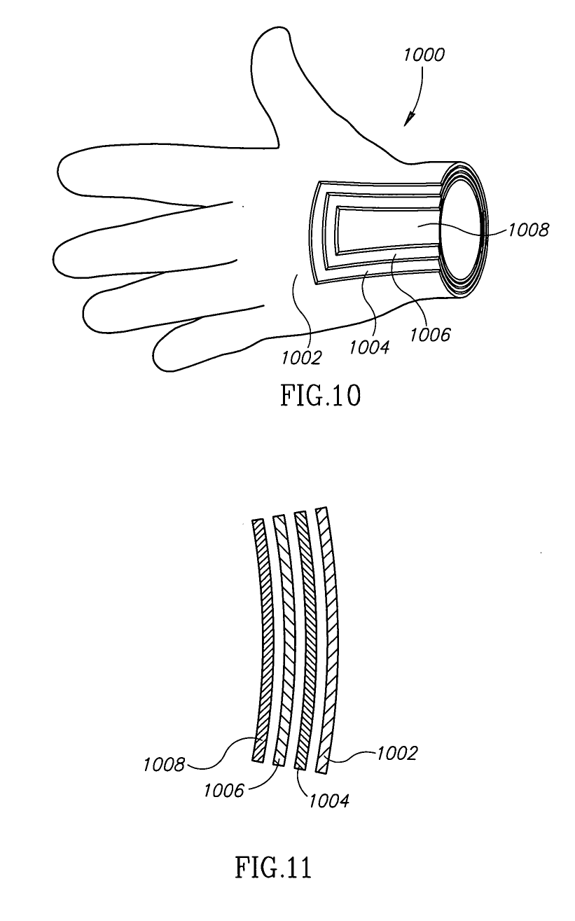

An optional inner layer 1008, directly against the skin, may reduce discomfort by keeping cooling layer 1004 and/or heating layer 1006 from direct contact with the skin, if cooling layer 1004 operates at too a low temperature, for example below 15 degrees C., and/or if heating layer 1006 operates at too high a temperature. Optionally, there is no inner layer 1008, and optionally at least the innermost of cooling layer 1004 and heating layer 1006 operates at a temperature that does not cause much discomfort when it comes into contact with skin. FIG. 11 is a more detailed view showing the four layers 1002, 1004, 1006 and 1008, in a cross-section of glove 1000.

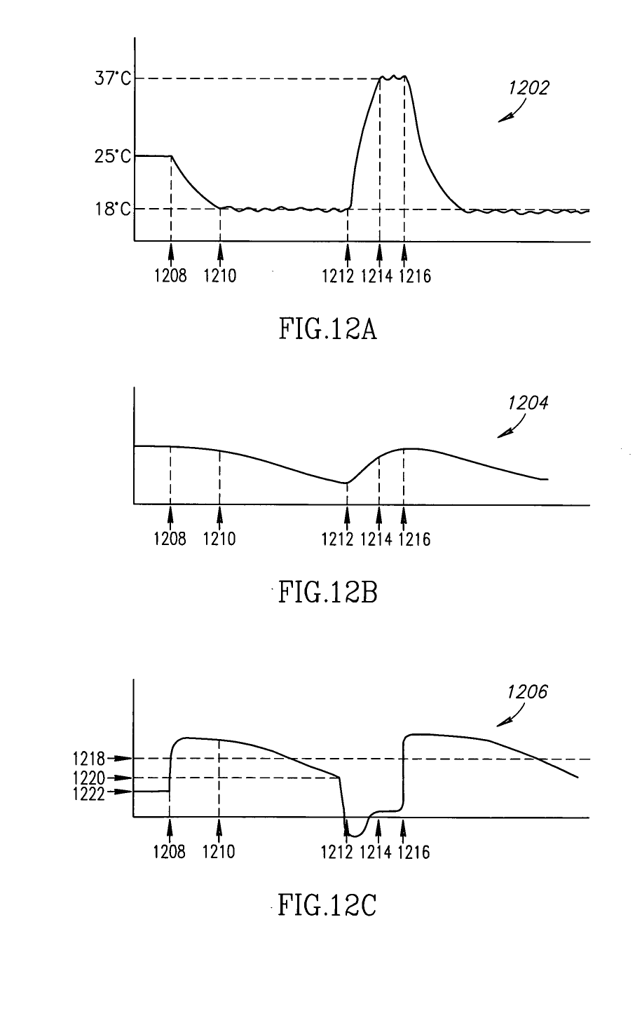

FIG. 12A shows a plot 1202 of skin temperature, FIG. 12B shows a plot 1204 of blood flow, and FIG. 12C shows a plot 1206 of heat loss rate, all as a function of time, for an exemplary method in which heating is alternated with cooling. The skin temperature is given in degrees Celsius, while the blood flow and heat loss rate are given in arbitrary relative units. Heat loss rates, averaged over time, for a single glove, have been found to be between about 50 and 400 kilocalories per hour, for example, in tests done by the inventors, as listed below in Table 1. Total blood flow rates through one hand, for example, are typically on the order of 100 to a few hundred milliliters per minute.

Initially, starting at time 1208, the skin is cooled from its ambient value of 25 degrees until it reaches 18 degrees at time 1210, and feedback is used to maintain the skin temperature at close to 18 degrees (schematically represented in plot 1200 as small oscillations around 18 degrees) until time 1212, about three to ten minutes after time 1208. A skin temperature of 18 degrees is optionally used at this time, because it is cool enough to remove heat from the body fairly effectively, but not so cool that it is uncomfortable, and/or because it is not so cool that it causes vasoconstriction too rapidly. The blood flow is initially high, and the heat loss rate is also high, approximately proportional to the blood flow times the difference between the temperature of the blood (at 37 degrees) and the skin temperature, once equilibrium is reached. After a few minutes, the capillaries begin to constrict as a result of the cold, and blood flow decreases, so the heat loss rate decreases as well, and by time 1212, the heat loss rate is significantly less than it was at time 1210. At this point, the cooling device is used as a heating device, heating the skin until it reaches 37 degrees, at time 1214, and feedback is used to maintain the skin at 37 degrees, until time 1216, about one to two minutes, or as much as ten minutes, after time 1212. Because this is the same as the temperature of the blood, the heat loss rate is almost zero during this period, but heat does not flow into the body either, except initially when the skin temperature is still increasing (indicated as a negative heat loss rate in plot 1206). As the skin is heated, the capillaries dilate, and the blood flow rate starts to recover, reaching its initial value by time 1216. The skin is then cooled again, with temperature, blood flow, and heat loss repeating the values seen between times 1208 and 1216. Although the heat loss rate is almost zero during the period between times 1214 and 1216, the average heat loss rate 1218 over the whole cycle is greater than the heat loss rate 1220 at time 1212 (which is somewhat above the equilibrium value that would be achieved if the skin were maintained at 18 degrees without intermittent heating), and the average heat loss rate 1218 is greater than the initial heat loss rate 1222 at time 1208 (the ambient heat loss rate if no cooling or heating were done). If the skin temperature were maintained indefinitely at 18 degrees, then the heat loss rate would asymptotically approach a level that is only slightly higher than the initial heat loss rate 1222, since the vasoconstriction of the capillaries is designed to conserve heat when the skin is cooled.

It should be noted that during the period between times 1214 and 1216, even though little or no heat is being lost to the cooling system, the relatively high 37 degree temperature of the skin in contact with the cooling system may induce the body to lose more heat elsewhere, for example by increasing blood flow and sweating. The total heat loss induced by the cooling system, including this indirect effect, may be greater than the direct heat loss through the cooling system shown in FIG. 12C.

Optionally, in using the thermal cycling method shown in FIGS. 12A-12C, the switch from cooling to heating, at time 1212, and/or the switch from heating to cooling, at time 1216, occur at fixed times. Alternatively, the switch from cooling to heating occurs when a direct measurement of cooling power (for example fluid flow rate times the difference between outlet and inlet temperature of the fluid), and/or a measurement of blood flow, indicates that cooling has become relatively ineffective. Optionally, the switch from heating to cooling occurs when a measurement of blood flow indicates that local capillaries are substantially dilated. Optionally, a switch from heating to cooling is temporarily made to directly measure the cooling rate, but cooling is not maintained unless the measurement shows a sufficiently high cooling rate.

Tests done by the inventors, in which subjects did not wear a cooling glove, but put their hands in cool water, or alternately in cool and warm water, suggest that thermal cycling can increase the average heat loss rate by a factor of two or more, compared to using only cooling.

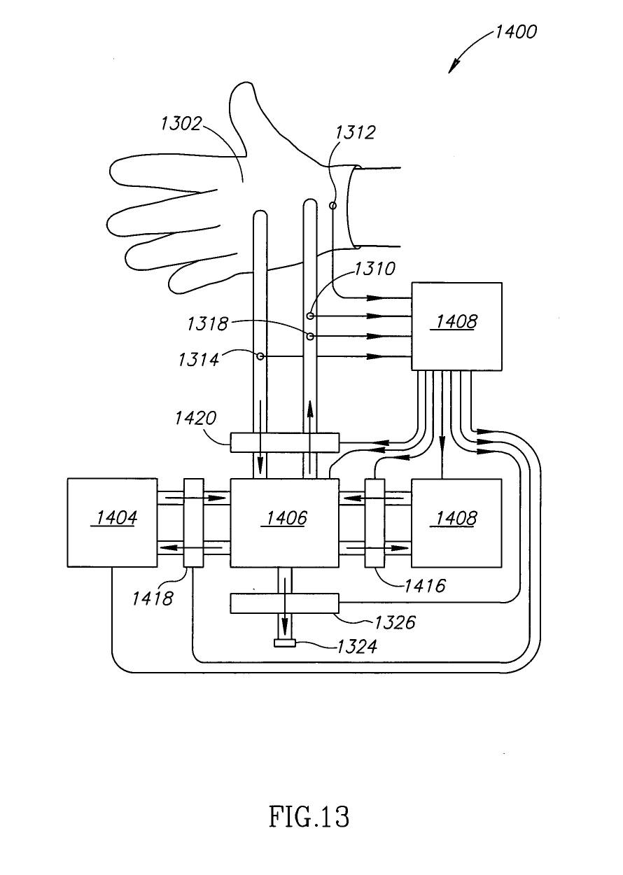

FIG. 13 schematically shows a cooling system 1400 which uses temperature cycling, in accordance with an exemplary embodiment of the invention. A pump 1406 pumps fluid through a glove 1302. A cooling unit 1404, and a heating unit 1408 supply fluid to the pump. Valves 1418 and 1416 respectively regulate the rate at which cooled fluid and heated fluid are supplied to the pump, and hence to the glove. Valve or valves 1420 optionally regulate the total rate at which fluid is pumped through the glove, and optionally pump 1406 can be controlled directly to regulate the flow rate of fluid through the glove. A valve 1326 optionally allows fluid to be drained out of the system through drain 1324, and optionally fluid can also be added to the system, for example through the cooling and heating units.

A control unit 1408 optionally receives temperature data from sensors 1310, 1312, and 1314, respectively at the inlet to the glove, at the skin of the hand inside the glove, and at the outlet to the glove. Control unit 1408 also receives flow data from sensor 1318. Control unit 1408 optionally controls any of the heating unit, the cooling unit, the pump, and the valves, in order to control the temperature and flow rate of fluid through the glove. The control unit optionally follows an algorithm which allows efficient cooling of the hand and little or no discomfort to the subject, optionally using thermal cycling to do this. The algorithm, for example, is similar to any of the algorithms described in the description of FIGS. 12A-12C.

In some embodiments of the invention, a cooling system is used in which two or more different parts of the body are cooled at different rates, or in which one part of the body is heated while another part of the body is cooled. Feedback loops, such as those described above for cooling system 1400 in FIG. 13, optionally use data pertaining to one part of the body to control the cooling or heating of the same and/or a different part of the body. Such a system has the potential advantage that it can make use of the fact that the body's own thermoregulatory systems use different parts of the body in different ways. For example, perhaps because maintaining the brain at a constant temperature is most important for proper functioning of the body, the hypothalamus exerts some systemic control over heating and cooling mechanisms throughout the body, in response to local temperatures in the brain. Heating the face, and hence the brain, while cooling the rest of the body, could cause the hypothalamus to suppress vasostriction and/or shivering in the trunk, and/or increase sweating in the trunk, allowing the core temperature of the trunk to fall somewhat below the normal core temperature of 37 degrees C., in order to keep the brain temperature at 37 degrees C. If heating of the face then stops, the hypothalamus may then trigger an increase in metabolism in the trunk, burning calories, in order to bring the core temperature of the trunk back up to 37 degrees C. Since the heat capacity of the trunk is greater than the heat capacity of the head, the net result may be a loss of heat from the body, and a resultant burning of stored fat.

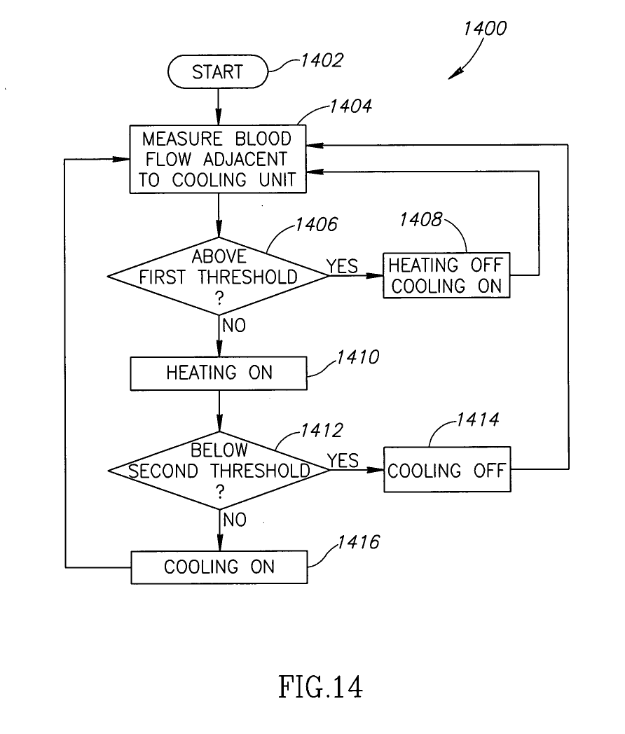

FIG. 14 shows a flowchart 1400 for an exemplary control loop which maintains heating of one part of the body, for example the face, with a heating unit, and cooling of another part of the body, for example the abdomen, with a cooling unit, at a level which may lead to a net loss of heat. The process starts at 1402. At 1404, blood flow rate adjacent to the cooling unit is measured, to determine the degree of vasoconstriction or vasodilation. At 1406, the measured blood flow rate is compared to a first threshold. If the measured blood flow rate is greater than the first threshold, then the heating unit is turned off, or kept off, and the cooling unit is turned on, or kept on, at 1408, the blood flow rate is measured again, at 1404. If the measured blood flow rate is less the first threshold, then the heating unit is turned on, or kept on, at 1410. Then, at 1412, the blood flow rate is compared to a second threshold, lower than the first threshold. If the blood flow rate is below the second threshold, then at 1414 the cooling unit is turned off, or kept off, and the blood flow rate is measured again at 1404. If the blood flow rate is above the second threshold, which means that it is between the first and second threshold, then the cooling unit is turned on or kept on, and the blood flow rate is measured again at 1404.

It should be noted that the control algorithm controlling the heating and cooling units treats the heating unit and cooling unit differently. When the heating unit is on, the cooling unit can be either on or off, and when the cooling unit is on, the heating unit can be either on or off. In some embodiments of the invention, whether or not there is a heating unit, there are two cooling units, which cool different parts of the body, and which are controlled by a control algorithm that treats the two cooling units differently. In some embodiments of the invention, the heating and/or cooling units are not limited to a single “on” state and an “off” state, but can operate at a plurality of different “on” levels, controlled by a control algorithm.

If the first and second thresholds are chosen properly, and the heating and cooling units have adequate heating and cooling rates, then the feedback loop shown in flow diagram 1400 will ideally result in duty cycles of the heating and cooling units which cause a net loss of heat from the body. The heating unit will heat the face, for example, sufficiently to maintain a relatively high level of vasodilation in the abdomen, for example, which will result in more cooling of the abdomen than heating of the face. But the heating will not be much more than necessary to maintain this high level of vasodilation in the abdomen, so that the body is not unnecessarily heated. The optimal values of the two thresholds are optionally determined by tests with a particular subject, since these values may differ for different subjects. Additionally or alternatively, guidelines for the values of the thresholds, and/or for the capacities of the heating and cooling units, may be based on a series of tests done on subjects with different body types, different genders, different base metabolisms, etc., to determine what works best for each subject.

Other ways to implement the feedback loop in flowchart 1400 will be apparent to those skilled in the art of control theory.

In some embodiments of the invention, a feedback loop similar to that shown in flowchart 1400 is used, but only with a heating unit, not with a cooling unit. Instead of active cooling, passive cooling occurs, because the heating of one part of the body, for example the face or another part of the head, induces the body's thermoregulatory mechanism to cause another part of the body to lose heat at an increased rate, due for example to vasodilation and/or sweating. Measuring an indication of this increased heat loss in the other part of the body, for example blood flow rate, or rate of sweating, or a combination of such indications, provides the feedback for controlling the heating unit, just as measuring blood flow rate adjacent to the cooling unit provides the feedback for controlling the heating unit in flowchart 1400. If the increased heat loss from the other part of the body is greater, averaged over a period of time, than the heating rate of the part of the body that is heated, then there will be a net loss of heat from the body, in spite of the fact that no active cooling is done.

As an alternative to the process shown in FIG. 14, the face, or another part of the body that has a systemic effect on vasoconstriction and vasodilation, may be cooled, resulting in vasoconstriction and/or an increase in heat generated in the trunk. If the trunk is not being cooled, this may cause the core temperature in the trunk to rise above normal core body temperature, nominally 37 degrees C. If the cooling of the face then stops, then the trunk will try to return to normal body temperature, by some combination of a decrease in heat generated, increased sweating, and vasodilation. Depending on the relative importance of each of these factors, during the period when the face is cooled and the period when the face is not cooled the net result may be an increase heat lost from the body, even more than the heat removed from the face during the cooling period.

Whether this procedure results in such an enhanced loss of heat, and whether it is more or less effective than the procedure shown in FIG. 14, or simple cooling of the trunk, may depend on characteristics of the subject. Optionally, tests are done to determine the best method of inducing heat loss from each subject, and/or a method is chosen based on tests done previously with subjects of different body type, base metabolism, gender, etc. Optionally, a single system may be programmed to use any of a variety of methods that may be chosen.

In some embodiments of the invention, a more sensitive part of the body is heated in order to prevent shivering or vasoconstriction, while a greater amount of heat is removed from a less sensitive part of the body, thereby preventing discomfort, or preventing vasoconstriction of the cooled part of the body. For example, the subject's buttocks are cooled by a cooling element in the seat of a chair, or the seat of an exercise bike, another part of the body is heated, for example the area of the spine.

Optionally, in order to cool one part of the body while heating another part, the heat removed from the cooled part of the body is directed to the heated part of the body. For example, if a refrigeration unit is used to cool a circulating cooling fluid, as in FIGS. 2A-2C or FIG. 2E, then waste heat from the refrigeration unit is directed to the heated part of the body, optionally in the form of blowing air, or a circulating heated fluid. If Peltier cooling elements are used, as in FIG. 2D or FIG. 4A, then heat produced by the Peltier units is optionally conducted to a radiating element which is positioned so that it convectively heats air which rises to the heated part of the body, for example the face. In the case of FIG. 2D, heat from the necklace optionally rises convectively, to heat the face, while cooling the upper chest. If the Peltier units are cooled by forced convection, as in FIG. 4C, the heated air is optionally directed to a part of the body that is to be heated, for example the face.

In some embodiments of the invention, the cooling system is used while sleeping, or right after eating a meal. If used in this way, the lowering of blood glucose levels associated with the increased metabolism may be less likely to stimulate the subject's appetite, and induce the subject to eat more food to replace the calories that are lost. Optionally, the cooling system is used in conjunction with one or more other weight loss methods, for example a diet, appetite suppressants, hormones which increase metabolism, or an exercise regimen. Using the cooling system together with other weight loss methods may make it less likely that the subject will consume enough extra calories to compensate for the heat removed by the cooling system, and/or may make it more likely that the subject will maintain a lower weight if use of the cooling system is discontinued. Optionally, the cooling device is used during exercise. However, if used during strenuous exercise, the cooling patch is preferably not used on the chest or the abdomen or the thighs, where it might impede loss of the heat that is generated in the core of the body by the exercise, but is used on peripheral regions, such as the hands or feet, particularly if the exercise does not impede blood circulation to these peripheral regions. A study by Jacobs et al, in European Journal of Applied Physiology 54: 35-39, 1985, the disclosure of which is incorporated herein by reference, shows that cooling the body is particularly effective at inducing weight loss if used shortly before exercising. There is also evidence that removing heat from the body is most effective for weight loss if done during light exercise, for example during normal activities during the day. For that purpose, a cooling patch may be advantageously worn under the clothing, in an unobtrusive location that will not interfere with normal activity.

Table 1 presents experimental results from three test runs with a prototype cooling device in a glove, on only one hand. The heat loss was calculated by multiplying the temperature rise T of the water by the flow rate in cc/sec and the run time in seconds, and dividing by 1000 to convert calories to kilocalories.

TABLE 1

| | Inlet | Outlet | Flow rate | Run time | | Heat lost | Heat lost |
|---|---|---|---|---|---|---|---|
| Expt. # | Temp. | Temp. | (cc/sec) | (minutes) | T (C.) | (kcal) | per hour |
| 1 | 15.8 C. | 21.0 C. | 10 | 50 | 5.2 | 156.0 | 187.2 |
| 2 | 15.7 C. | 20.7 C. | 10 | 25 | 5.0 | 75.0 | 180.0 |
| 3 | 11.5 C. | 22.08 C. | 10 | 30 | 10.58 | 190.4 | 380.9 |

The subject had a slight sensation of shivering throughout these test runs, but did not feel uncomfortable. For comparison, it should be noted that the average heat loss rate for a resting adult without the cooling device operating is about 80 kilocalories per hour, so the cooling device more than doubles the heat loss rate, and in the case of experiment #3, nearly multiplies it by 5. Even higher rates can be expected if more than one cooling device is used, for example a glove on each hand. These tests show the feasibility of losing several hundred calories, above the normal heat loss, in a reasonable time, without causing discomfort.

In other tests, the subject immersed one hand in water at 18 degrees Celsius, a comfortable temperature, and lost 10 to 40 kilocalories of heat per hour, typically 15 to 30 kilocalories per hour. It is estimated that with two hands and two feet immersed in water at 18 degrees, between 50 and 100 kilocalories could be lost per hour. The heat loss rates were lower than for the tests shown in Table 1, because the water was at a higher temperature, and it was not pumped past the hand. For comparison, it should be noted that in fast walking, about 400 kilocalories are consumed per hour.

The invention has been described in the context of the best mode for carrying it out. It should be understood that not all features shown in the drawing or described in the associated text may be present in an actual device, in accordance with some embodiments of the invention. Furthermore, variations on the method and apparatus shown are included within the scope of the invention, which is limited only by the claims. Also, features of one embodiment may be provided in conjunction with features of a different embodiment of the invention. As used herein, the terms “have”, “include” and “comprise” or their conjugates mean “including but not limited to.”

1. A method for increasing metabolism of a subject's body in order to lose weight, comprising:
a) contacting a part of the body with a cooling element to remove heat from the body; and
b) repeating or continuing (a) so as to remove enough heat in total to lose at least 1 kilogram of body weight.

2. A method according to claim 1, wherein the cooling element is worn by the subject.

3. (canceled)

4. A method according to claim 1, wherein the cooling element is comprised in furniture used by the subject.

5-6. (canceled)

7. A method according to claim 1, wherein the cooling element is comprised in exercise equipment, used by the subject for exercising while the cooling element removes heat from the subject's body.

8. A method according to claim 1, wherein removing heat from the body is at a great enough rate, over a long enough period at a great enough duty cycle, so that the subject's base metabolism increases by at least 10% for at least one day after the heat is removed.

9. A method according to claim 1, wherein contacting a part of the body comprises contacting an internal part of the body.

10. A method according to claim 9, also including performing an action on the body other than cooling.

11. A method according to claim 10, wherein the cooling element comprises fluid introduced into the subject's colon, and the action is colonic irrigation.

12. (canceled)

13. A method according to claim 1, wherein contacting comprising drawing the body and the cooling element together by vacuum.

14. A method according to claim 1, wherein contacting comprises contacting through a thermally conducting liquid.

15. A method according to claim 1, wherein the cooling element removes at least 10 kilocalories of heat within a period shorter than one hour.

16. (canceled)

17. A method according to claim 1, wherein the cooling system does not lower the core body temperature by more than 1 degree Celsius when the heat is removed from the body.

18. A method according to claim 1, also including actively controlling the rate of heat removal by the cooling element while it is in contact with the body, using a control algorithm.

19. A method according to claim 18, also including contacting a second part of the body with a second element, and actively controlling the second element to remove heat from the body or to add heat to the body, using the control algorithm, wherein the control algorithm treats the cooling element and the second element differently.

20. A method according to claim 19, wherein the heating or cooling the second part of the body has a systemic effect on the body's thermoregulatory mechanism.

21. A method according to claim 20, wherein the second part of the body is at least part of the head.

22. A method according to claim 18, also including choosing a tolerable level of discomfort, wherein actively controlling the rate of heat removal by the cooling element comprises actively controlling the rate of heat removal to avoid exceeding the tolerable level of discomfort.

23. A method according to claim 22, wherein choosing the tolerable level of discomfort is done at least within limits by the subject, in real time during the removal of heat from the body.

24. A method according to claim 18, wherein actively controlling the rate of heat removal comprises controlling the rate of heat removal to avoid damage to body tissue.

25. A method according to claim 1, also including reducing vasoconstriction during the removal of the heat from the body.

26. A method according to claim 25, wherein reducing vasoconstriction comprises causing vasodilation.

27-33. (canceled)

34. A method according to claim 25, wherein reducing vasoconstriction comprises heating the skin.

35. A method according to claim 34, wherein heating the skin is alternated with removing the heat, in such a way that there is a net removal of heat from the body.

36. A method for increasing metabolism of a body in order to lose weight, comprising:
a) causing vasodilation of peripheral blood vessels locally in a part of the body, thereby causing heat to be lost from the body, beyond the heat that would have been lost from the body in the same thermal environment in the absence of the vasodilation; and
b) repeating or continuing (a) so as to remove enough heat in total to lose at least 1 kilogram of body weight.

37. A method according to claim 36, wherein the vasodilation causes at least 10 kilocalories of heat to be lost from the body within a period shorter than one hour.

38. (canceled)

39. A method for increasing metabolism of a subject's body in order for the subject to lose weight, the method comprising:
a) heating a part of the body, causing a thermoregulatory mechanism of the body to increase heat loss from another part of the body;
b) measuring at least an indicator of the increased heat loss; and
c) repeatedly or continuously adjusting the rate of heating in response to a change in the measured indicator, in a direction opposite to the change in indicated heat loss;
wherein the increase in heat loss, averaged over a period of time, is greater than the rate of heating.

40. (canceled)

41. A method according to claim 39, wherein heating a part of the body comprises heating at least part of the head.

42. A cooling system for increasing metabolism of a body in order to lose weight without exceeding a chosen tolerable level of discomfort, the system comprising:
a) a cooling patch adapted to directly or indirectly contact the skin on a part of the body, which cooling patch removes heat from the body by cooling the skin; and
b) a controller adapted to actively control the rate at which the cooling patch removes heat from the body while it is in contact with a given area of the skin;
wherein the controller is adapted to control the cooling patch to remove the heat from the body using a feedback loop that regulates the rate of heat loss, or the level of discomfort, or both.

43. A cooling system according to claim 42, wherein the cooling patch is adapted to be worn by the subject.

44. (canceled)

45. A cooling system according to claim 42, wherein the cooling patch is comprised in furniture.

46-47. (canceled)

48. A cooling system according to claim 42, wherein the cooling patch is comprised in exercise equipment.

49. A cooling system according to claim 42, wherein the feedback loop regulates the rate of heat loss to be at least 10 kilocalories in less than one hour.

50. (canceled)

51. A cooling system according to claim 42, wherein the controller is adapted to control the cooling patch to remove the heat without lowering the core temperature of the body by more than 1 degree Celsius.

52. A cooling system according to claim 42, wherein the cooling patch is comprised in a glove, adapted to contact the skin of the hand.

53. A cooling system according to claim 42, also comprising:
a) a cooling fluid; and
b) a pump which circulates the cooling fluid through the cooling patch, thereby removing the heat from the body.

54. A cooling system according to claim 53, also including a detachable reservoir of a cooling material, adapted for cooling in advance, through which the cooling fluid also circulates.

55. A cooling system according to claim 53, also including a refrigeration unit which cools the cooling fluid when the cooling fluid passes through it, wherein the pump also circulates the cooling fluid through the refrigeration unit, thereby transferring the heat removed from the body to the refrigeration unit.

56. (canceled)

57. A cooling system according to claim 42, also including moving elements adapted to cause vasodilation of the skin cooled by the cooling patch, by vibrating or massaging.

58. A cooling system according to claim 42, also including electrodes and a source of electric current adapted to cause vasodilation of the skin cooled by the cooling patch, by electric stimulation.

59. A cooling system according to claim 42, also including a heating element adapted to cause vasodilation of the skin cooled by the cooling patch, by heating the skin alternately with the cooling of the skin.

60-61. (canceled)

62. A cooling system according to claim 59, wherein the controller is adapted to alternately control the cooling patch to cool the skin for a cooling interval, and control the heating element to heat the skin for a heating interval.

63. (canceled)

64. A cooling system according to claim 62, also including at least one sensor, wherein one or both of the cooling and heating interval depend on data from the at least one sensor.

65. A cooling system according to claim 42, wherein the controller controls the cooling patch to cool the skin at a rate that varies in time.

66. A cooling system according to claim 65, also including at least one sensor, wherein the rate is a function of time that depends on data from the at least one sensor.

67-68. (canceled)

69. A cooling system according to claim 64, wherein the at least one sensor senses blood flow.

70. A cooling system according to claim 64, also including a cooling fluid and a pump which circulates the cooling fluid through the cooling patch, thereby removing the heat from the body, wherein the at least one sensor senses a flow rate of the cooling fluid.

71. A cooling system according to claim 42, wherein the cooling patch is comprised in a seat used in a vehicle.

72. A cooling system according to claim 66, wherein the at least one sensor senses blood flow.

73. A method according to claim 1, wherein contacting a part of the body comprises contacting one or both of the trunk and the limbs.

74. A method according to claim 73, wherein contacting a part of the body comprises contacting one or more of the thighs, the lower legs, the buttocks, and the abdomen.

75. A system according to claim 42, comprising a display which displays one or more of an inlet temperature, an outlet temperature, a difference between inlet and outlet temperatures, a skin temperature, an elapsed time, a flow rate, a cumulative heat loss in kilocalories or in other units, and data of physiological interest.

76. A method according to claim 18, wherein actively controlling the level of heat removal comprises stopping the cooling, or changing the rate of cooling, or changing a duty cycle of cooling, at a condition set by the subject in advance.

77. A system according to claim 42, wherein the cooling patch is comprised within a chair, sofa, toilet seat, or seat of an exercise bike.

78. A method according to claim 1, also including controlling the rate of heat removal by the cooling element, by one or both of controlling a duty cycle of the cooling element and controlling a temperature of the cooling element.
